SEQUENCE LISTING (5 sequences)

SEQ ID NO: 1 - protein, 4 aa, Artificial Sequence
  Description of Artificial SequenceC-terminal PDZ-ligand (PL) of PAG
ITRL

SEQ ID NO: 2 - protein, 4 aa, Artificial Sequence
  Description of Artificial SequenceC-terminal PDZ-ligand (PL) of PAG C-ARA
IARA

SEQ ID NO: 3 - DNA, 24 bp, Artificial Sequence
  Description of Artificial Sequence oligonucleotide 736 SHF
tggatccttgaggagaagacggtg

SEQ ID NO: 4 - DNA, 26 bp, Artificial Sequence
  Description of Artificial Sequence oligonucleotide 737 shr
tgcaattgtcgtcggggtccagattc

SEQ ID NO: 5 - protein, 4 aa, Artificial Sequence
  Description of Artificial SequenceGLGF repeat PDZ domain
GLGF

(19) United States
(12) Patent Application Publication
(10) Pub. No.: US 2010/0215677 A1
(43) Pub. Date: Aug. 26, 2010

(54) PDZ DOMAIN INTERACTIONS AND LIPID RAFTS

(75) Inventors: Peter S. Lu, Palo Alto, CA (US); Chamorro Somoza Diaz-Sarmiento, Mountain View, CA (US); Brian Seed, Boston, MA (US); Ramnik Xavier, Boston, MA (US); Bryan Allen Irving, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Arbor Vita Corporation, Fremont, CA (US); The General Hospital Corporation, Boston, MA (US)

(21) Appl. No.: 12/770,682

(22) Filed: Apr. 29, 2010

Related U.S. Application Data
(63) Continuation of application No. 11/674,034, filed on Feb. 12, 2007, now abandoned.
Application No. 11/674,034 is a continuation of application No. 10/080,273, filed on Feb. 19, 2002, now abandoned.
(60) Provisional application No. 60/269,523, filed on Feb. 16, 2001.
(60) Provisional application No. 60/269,522, filed on Feb. 16, 2001.
(60) Provisional application No. 60/269,694, filed on Feb. 16, 2001.

(51) Int. Cl. A61K 39/00; G01N 33/53; C12N 5/07; A61P 37/00; A61P 3/10; A61P 19/02; A61P 17/06; A61P 35/02; A61P 35/00; A61P 1/00; C07K 2/00; C07K 14/00

(52) U.S. Cl. 424/185.1; 435/7.1; 435/375; 424/184.1; 424/192.1; 530/300; 530/324

(57) ABSTRACT

Methods for modulating immune cell signaling are provided. In general such methods involve modulating an interaction between a PDZ protein and a PDZ ligand protein whose interaction affects the composition and/or distribution of lipid rafts in an immune cell. Modulators that enhance or inhibit such interactions are also disclosed, as well as methods of screening for such modulators.

### CROSS-REFERENCE TO RELATED APPLICATIONS

This application is a Continuation of U.S. patent application Ser. No. 10/080,273, filed Feb. 19, 2002, which claims the benefit of U.S. Provisional Application Nos. 60/269,523; 60/269,522; and 60/269,694, all filed Feb. 16, 2001, each of which are incorporated herein by reference in their entirety for all purposes.

### BACKGROUND

Engagement of the T cell antigen receptor (TCR) by antigen presentation initiates a sensitive, highly regulated response that relies on the coordinated action of a large number of signaling proteins. Recent evidence has shown that extensive rearrangements of membrane and cytoskeletal elements attend the activation response, and that compounds that disrupt the organization or localization of these elements interfere with antigen recognition (Acuto and Cantrell, 2000; Bromley et al., 2001; Bunnell et al., 2001; Dustin et al., 1998; Grakoui et al., 1999; Wulfing and Davis, 1998). A similar phenomenon appears to be involved in B cells.

The plasma membrane of lymphocytes is believed to have a variegated structure comprising discrete microdomains or “lipid rafts” dispersed in a larger sea of phospholipids (see, e.g., Simons and Toomrc, 2000, Nature Reviews Molecular Cell Biology 1:31-39; Schutz et al., 2000, EMBO J. 19:892-901; Rietveld et al., 1998, Biochim. Biophys. Acta 1376:467-79; Pralle et al., 2000, J. Cell Biol. 148:997-1008). Lipid rafts are composed primarily of glycosphingolipids and cholesterol and were first identified based on their insolubility in some nonionic detergents such as Triton X-100, with the tighter packing properties of sphingolipids relative to phospholipids likely accounting for this phenomenon (3). The insolubility and buoyant properties of rafts have enabled their isolation via density centrifugation. In addition to possessing distinct lipid composition, lipid rafts are enriched in glycosylphosphatidyl inositol linked proteins, as well as a variety of cytoplasmic and transmembrane proteins that localize to lipid rafts via post-translational acylations (2, 4). The unique composition of the lipid rafts provide cells such as lymphocytes a means to partition and regulate the dynamics of the select subset of proteins that reside in the rafts (2). For example, the finding that lipid rafts are enriched in certain proteins that couple surface receptors to intracellular signal transduction and that lipid rafts coalesce at sites of receptor engagement indicate that the proteins play a role in the capacity of a cell to interpret and translate extracellular cues. Thus, for instance, in lymphocytes the dispersal of the lipid rafts appears to attenuate the antigen response.

Antigen-dependent activation appears to be initiated by phosphorylation of the intracellular domains of the TCR by Src family kinases, amplified by the recruitment and activation of Syk family kinases, and sustained by molecular reorganizations that permit multiple levels of regulatory control. During the activation process a structured interface is formed between the antigen presenting and responding cell that requires the energy-dependent coordinated movement of large supramolecular aggregates.

Under certain conditions receptor engagement leads to the assembly of a characteristic supramolecular activation complex (SMAC) on the T lymphocyte side of the interface. The SMAC can be divided into two concentrically organized subcomplexes: a central supramolecular activation complex (c-SMAC) and a peripheral supramolecular activation complex (p-SMAC) (Monks et al., 1997; and Monks et al., 1998). Protein kinase C isoform θ (PKC-θ) is concentrated in the c-SMAC, whereas LFA-1 is concentrically arrayed around the PKC-θ-rich zone in the p-SMAC (Monks et al., 1997). Although this organization is not detected when powerful activating stimuli are applied (Monks et al., 1997), it seems likely that the microscopic features that give rise to the visible SMAC complexes are nonetheless present under a variety of conditions leading to T cell activation.

However, to date, a specific mechanism by which membrane microdomains/lipid rafts and signaling molecules might undergo coalescence or translocation has not been described. The ability to regulate the protein constituents of lipid rafts and their cellular distribution, however, would be a powerful tool in modulating a number of receptor-mediated cellular processes given the role the lipid rafts appear to play in signal transduction.

### SUMMARY

The present inventors have discovered that interactions between certain PDZ proteins and their cognate ligand proteins such as PL proteins play a role in the organization, assembly and disruption of protein complexes within lipid rafts of immune cells. Furthermore, they have found that such interactions play a role in the redistribution of lipid rafts that occurs following immune receptor stimulation. Because such events and the formation of a structured interface between antigen-presenting and responding cells are involved in the regulation of immune cell signaling, modulation of the PDZ/cognate ligand protein interaction can be utilized to modulate immune cell signaling. Thus, a variety of methods of modulating immune cell signaling, modulators and composition that affect immune cell signaling and methods for screening for such modulators are provided herein.

For example, certain methods for modulating immune cell signaling generally involve modulating an interaction between a PDZ protein and a PDZ ligand protein (a PL protein), which interaction affects the composition and/or distribution of lipid rafts in an immune cell, and whereby such modulation alters immune cell signaling. Some of the interactions that have been identified as playing a role in affecting lipid raft composition and/or distribution are summarized in Tables II and III infra Examples of PDZ proteins that are involved in such processes include, but are not limited to hDlg, SHANK1, SHANK3, EBP-50, CASK, KIAA0807, TIP1, PSD-95, Pick 1, CNK, GRIP and DVL-2. Exemplary PL proteins involved in such interactions include, but are not limited to, PAG, LPAP, ITK, DNAM-1, Shroom, PTEN, BLR-1, fyn and Na+/Pi transporter.

In certain methods, interactions between specific PDZ proteins and PL proteins are modulated. Examples of such interactions are those in which: (a) the PDZ protein is SHANK1 or SHANK3 and the PL protein is PAG, LPAP, ITK, DNAM-1, Shroom, PTEN, BLR-1 or fyn; (b) the PDZ protein is TIP1 and the PL protein is LPAP or PAG; (c) the PDZ protein is KIAA0807 and the PL protein is PAG or LPAP; (d) the PDZ protein is EBP-50 and the PL is PAG or LPAP or BLR-1; or (e) the PDZ protein is SHANK3 or EBP-50 and the PL protein is Na+/Pi transporter.

Modulation of the PDZ protein and cognate ligand protein interactions that are disclosed herein can be used in the therapeutic or prophylactic treatment of patients (either humans or non-humans) that are suffering from an immune disorder. Such methods involve administering a compound to the patient, wherein the compound is one that inhibits or enhances interaction between the PDZ protein and the PL protein and is administered in an amount effective to treat the immune disorder. Such methods can be utilized to treat various autoimmune disorders for example, but can also be used to treat non-autoimmune disorders (e.g., lymphoma and leukemia).

Modulators of immune cell signaling are also provided. In general such compounds modulate binding of a PDZ protein and a PDZ ligand protein (a PL protein), wherein the modulator inhibits or enhances binding of a PDZ domain polypeptide and a PL domain polypeptide, and wherein (i) the PDZ domain polypeptide comprises at least a partial sequence of the PDZ protein and the PL domain polypeptide comprises at least a partial sequence of the PL protein; and (ii) the PDZ protein and the PL protein are proteins which in an immune cell can interact with one another to affect the composition and/or distribution of lipid rafts in the immune cell. Both agonists and antagonists of the interaction are provided. Certain antagonists are a polypeptide or fusion polypeptide comprising a sequence that is from 2 to about 20 residues of a C-terminal sequence of the PL protein involved in the interaction. Other antagonists are a polypeptide or fusion polypeptide comprising a sequence that is from 2 to about 100 (or 20 to 100) residues of the PDZ domain of the PDZ protein. Still other antagonists are peptides or small molecule mimetics of the foregoing polypeptides or fusion polypeptides. The modulators can be ones that inhibit or enhance the binding of the PDZ and PL proteins listed in Tables II and III, as well as those specific interactions mentioned supra.

Methods of screening for modulators are also provided. In general certain such methods involve identifying a compound that modulates interaction between a PDZ protein and a PDZ ligand protein, wherein the PDZ protein and the PL protein are proteins which in an immune cell can interact with one another to affect the composition and/or distribution of lipid rafts in the immune cell. In some instances, the identification process more specifically involves contacting a PDZ domain polypeptide that comprises at least a partial sequence of the PDZ protein and a PL domain polypeptide that comprises at least a partial sequence of the PL protein in the presence of the compound. One then determines whether there is a statistically significant difference in the amount of complex formed between the PDZ domain polypeptide and the PL domain polypeptide in the presence of the compound as compared to the amount of the complex formed in the absence of the compound, a statistically significant difference being an indication that the compound is a modulator of immune cell signaling. Such screening methods can be performed to identify modulators for any of the PDZ/PL interactions described in Tables II and III or the specific interactions listed above, for example.

The modulators having the structure described above or identified by the screening methods that are provided can be formulated as a pharmaceutical composition that comprises the modulator and a pharmaceutically acceptable carrier. Thus, also disclosed herein is the use of a modulator of the binding of a PDZ protein and a cognate ligand protein (e.g., a PL protein) in the preparation of a medicament for treatment of an immune disease, wherein the PDZ protein and the PL protein are proteins which in an immune cell can interact with one another to affect the composition and/or distribution of lipid rafts in the immune cell.

### BRIEF DESCRIPTION OF THE DRAWINGS

FIG. 1 is a schematic of PAG and certain mutants described herein. The cytoplasmic domain of PAG contains several sites for tyrosine phosphorylation, one of which binds the inhibitory kinase, csk. The amino acids comprising the C-terminal PDZ-ligand (PL) of PAG are shown (-ITRL), in addition to those of the mutants constructed: PAG C-ARA(-IARA) and PAG ΔPL(-I). A FLAG epitope was introduced downstream of the CD8 leader sequence to facilitate expression analysis.

FIGS. 2A and 2B are charts showing enhanced inhibition by PAG with mutation of its PDZ-binding motif Jurkat T cells, in which a β-galactosidase reporter gene under the control of the NFAT binding site had been stably integrated, were transiently transfected with the designated PAG constructs. A truncated form of the DR6 tumor necrosis factor receptor was used as a control in the experiment. Twenty-four hours after transfection, cells were stimulated with anti-TCR antibodies (FIG. 2A) or Ionomycin (a calcium ionophore that activates T cells and causes calcium flux)+PMA (Phorbol 12-myristate 13-acetate) (FIG. 2B) for 6 hours, then analyzed for (3-galactosidase activity and expression of the N-terminal FLAG epitope by flow cytometry. Results are expressed as the percentage of activated cells within the three designated populations: Flag (−) or untransfected cells, and those that expressed either low-intermediate (1-2 logs by FACS expression), or high levels of the transfected proteins (2+ logs fluorescence), Flag (+).

FIG. 3 is a schematic illustrating a proposal for PAG function in T cell activation. As shown, the proposal is that PAG (or csk-binding protein) negatively regulates src-family kinases that are involved in the initial stages of activating T and B cells. Phosphorylation of the C-terminal tyrosine residue of the src-family kinases inactivates the kinase by causing the enzyme to fold in an SH2-phosphotyrosine-dependent way such that the active site is not available to substrates. In the resting state of T cells, PAG inhibits srk kinases such as lck by binding csk and positioning it to phosphorylate lck, the kinase responsible for initiating a T cell response. Activation of a T-cell causes dephosphorylation of PAG which in turn results in the release of csk. The release of csk allows the phosphatase CD45 to dephosphorylate and activate lck, which in turn can activate the T cell. As PAG contains a PL domain, it is expected that the activity of PAG can be regulated by a PDZ domain-containing protein such as KIAA807, Shank or EBP-50.

FIGS. 4A and 4B illustrate major domains and interactions involving Shank 1, Shank2 and Shank3 proteins. FIG. 4A is a domain map showing interactions between the Shank1, Shank2 and Shank3 proteins with proteins such as spectrin, GRIP, GKAP, Horner and Cortactin1. Domains are listed below the line, except for the multimerization domain. Potential interacting proteins identified for an individual Shank protein are listed above the lines. FIG. 4B is a schematic of potential interactions involving PAG and the Shank proteins for regulating raft involvement in T cell activation. A PDZ domain-containing protein such as Shank binds PAG (see infra), which is known to localize to lipid rafts. Shank1 interacts with the cytoskeleton and may be involved in the reorganization of lipid rafts to the immune synapse upon activation by an antigen presenting cell. Other PDZ domain containing proteins could fulfill this link between rafts and the cytoskeleton as well.

FIGS. 5A-I are binding plots of interactions between selected PL proteins and the PDZ domain containing proteins Shank1, Shank3 and EBP-50 (domain 1 and domain 2). G-assays (see Example 4) were performed using components listed for each panel (A-I), titrating the amount of ligand to obtain a range of binding. All data points are duplicate or triplicate, and error bars are included for all data points.

FIGS. 6A and 6B provide a summary of expression of PDZ proteins in T cells. FIG. 6A is a schematic diagram of the PDZ-containing proteins analyzed for expression in T cells. Abbreviations for the various domains contained within the proteins are as follows: PRO (proline rich region); PDZ (acronym for PSD-95; Disks Large, and Zona Occludens-1); SH3 (src-homology 3); 13 (actin-binding element); GK (guanylate kinase domain); CaM (Calmodulin kinase domain); ANK (ankyrin repeats); SAM (serial alpha motif); CRIC (conserved region in cnk); PH (pleckstrin homology domain). Expression is indicated by a “+” sign (expression observed) or a “−” sign, expression not observed. FIG. 6B includes Western blots showing which PDZ proteins and proteins associated with T cell activation are present in microdomains. T cells were unstimulated or stimulated with OKT3, and lysates fractionated into cytoplasmic (C), membrane (M), and DIG-detergent-insoluble glycolipid-enriched—(D) fractions, and analyzed by Western blotting with the indicated antibodies. LAT, Lck, PKCθ, Lfa-1, csk, hDLG and CASK all appear to be associated with rafts independent of activation of the T cell receptor. GADS and IQGAP appear to associate with rafts, but less strongly.

FIGS. 7A-7D show the structure of Discs Large (hDlg) and Western blots characterizing the expression of hDlg. FIG. 7A is a schematic representation of the domains within Discs Large. The modular domains and the identity of proteins known to associate with each domain are depicted. FIG. 7B is a Western blot showing that hDlg association with microdomains does not require Lck. The abbreviations have the following meanings: cytoplasmic fractions (C); membrane fractions (M); and Detergent-insoluble glycolipid-enriched fractions (D). The presence of hDlg was analyzed in Jurkat T cells and an lck-deficient Jurkat variant, Jcam1.1, by immunoblotting. FIG. 7C is a Western blot showing that T-cell activation promotes the association of membrane hDlg with the actin cytoskeleton. Jurkat T cells were left unstimulated or stimulated with OKT3 mAb, lysed, and the indicated cellular fractions (total, cytoplamic, and membrane), immunoprecipitated with anti-hDlg 1 antibody. The immunoprecipitates were analyzed by SDS-Page, followed by Western blotting with an actin-specific antibody. FIG. 7D shows another Western blot demonstrating that tyrosine phosphorylated proteins associate with hDlg upon stimulation of the TCR and CD28. Jurkat cells were stimulated with the indicated antibodies or H₂O₂ (pervanadate), lysed, and the hDlg-immunoprecipitates analyzed for phosphotyrosine-containing proteins by western blotting with mAb 4G10. The position of PLCγ1, hDlg and CD3ζ are indicated. The blot was reprobed with hDlg antibodies to confirm the presence of relatively comparable levels of hDlg in each immunoprecipitate.

FIG. 8 shows results of immunoprecipitation and Western blots to demonstrate that multiple domains of hDlg are required for interaction with Cbl. Fusion proteins containing the indicated regions of hDlg (see FIG. 11) were analyzed for their ability to bind cbl in lysates from Jurkat T cells. Quantities of each hDlg fusion and total levels of cbl are shown.

FIG. 9 shows that multiple signaling molecules associate with hDIg1 in T cells. Membrane (M+) and cytosolic fractions (M−) from CD3/CD28 stimulated Jurkat cells were immunoprecipitated with hDlg1 antibody, resolved by SDS-PAGE and immunoblotted with antibodies recognizing the proteins shown (listed to the left of each immunoblot). All of these molecules except Fyn and ZAP-70 associate with hDlg directly or indirectly; however, LFA-1 and CD3ζ appear to associate more with membrane-localized forms of hDLG after CD3/CD28 stimulation. The bands observed in the Fyn and ZAP-70 do not appear to be the expected size (indicated by arrows).

FIG. 10 includes a schematic of certain domains of Discs Large and includes a chart summarizing whether certain signaling proteins are interaction partners with hDlg in T cells. The detected interactions are designated with plus signs; proteins showing no interaction are indicated with minus signs.

FIG. 11 provides a schematic depiction of the GFP/Dlg fusion proteins used to delineate the minimal requirements for association with lck, CD3ζ, LAT, and Cbl. The names for the mutants derive from the regions that each protein contains. The Dlg fusions, expressed in Jurkat cells, were immunoprecipitated and their associations determined by Western blotting using antibodies specific to Lck, CD3ζ, LAT and Cbl. Positive interactions are designated with a plus.

FIGS. 12A-12C are charts showing that hDlg1 induces apoptosis in Jurkat T cells. Jurkat cells expressing SV40 Large T antigen were electroporated with vectors encoding hDlg1-GFP (FIGS. 12A and B), the internal deletion mutant, hDlg1NGK-GFP (consisting of residues 1-186, the N-terminus fused to 683-906, and the guanylate kinase domain), (FIGS. 12A and 12C), or GFP alone. GFP intensity was measured by flow cytometry. FIG. 12A shows Annexin V reactivity of Jurkat cells electroporated with hDlg1-GFP, NGK-GFP, or GFP. Cells were transfected with vectors expressing hDlg1-GFP, hDlg1NGK-GFP, CASK-GFP, or GFP and stained with phycoerythrin (PE)-conjugated Annexin V. The percentage of annexin positive, GFP positive cells was calculated as a fraction of the total GFP positive cells, and the contribution of spontaneous annexin reactivity (percentage of annexin positive, GFP positive cells among cells transfected with GFP alone, approximately 10%) subtracted from the total. The Dlg-mediated apoptosis observed was refractory to zVAD, an inhibitor of conventional apoptosis. In another set of experiments, Jurkat cells were transfected with GFP alone, GFP and hDlg (FIG. 12B), or GFP and the hDlg internal deletion mutant, NGK (FIG. 12C), then analyzed for the percentage of live cells expressing GFP by flow cytometry. HDlg tranfection induced apoptosis in Jurkat cells, and the NGK deletion only reduced this effect mildly.

FIG. 13 provides a schematic illustration of various hDlg mutants to delineate the domains involved in mediating the cell death response. As in FIGS. 12A-12C, Jurkat cells were transfected with GFP in addition to one of the indicated hDlg fusion proteins. The percentage of cells surviving (as monitored by the % GFP positive pool) is presented.

FIG. 14 is a chart of fluorescence intensity as a function of time showing that expression of hDlg attenuates the TCR-mediated mobilization of calcium. Jurkat T cells untransfected (OKT3) or transfected with hDlg (hDlg) were loaded with a calcium-sensitive fluorescent dye and stimulated with OKT3 antibody. The TCR-mediated calcium responses are shown.

FIGS. 15A and 15B summarize certain protein interactions with CASK. FIG. 15A is a schematic representation of CASK and depicts certain partners that interact with various domains. Domains are indicated above the line and interactions listed below. FIG. 15B is a schematic representation of the assay used to define the interaction requirements for CASK association with the Cdc42/rac GTPase. An N-terminal FLAG-tagged version of Cdc42/rac was co-transfected with a series of C-terminal Au1-tagged CASK deletion mutants. Cdc42/rac was precipitated via the FLAG epitope and associations monitored by immunoblotting with an Au1-specific mAb.

FIGS. 16A and 16B show results of CASK interaction data in Jurkat and 293T cells. FIG. 16A includes Western blots showing CASK interactions in Jurkat T cells. Jurkat cells were unstimulated (−) or stimulated with OKT3 (+), lysed, and fractionated into cytoplasmic (C) and membrane (M) fractions. CASK was immunoprecipitated from these fractions and its association with the indicated proteins analyzed by Western blot using antibodies specific to the proteins listed at the left or right of each Western blot. FIG. 16B summarizes certain CASK interactions in 293T cells. Au1 epitope-tagged CASK was co-transfected into 293T cells with ZAP-70, hDlg1, cbl, or vav. Total cell lysates (TL) or anti-Au1 immunoprecipitates (ip) were analyzed by immunoblotting with the indicated antibodies. ZAP-70, vav and hDlg appear to co-immunoprecipitate with CASK whereas Cbl does not.

FIG. 17 shows activation-dependent association of signaling molecules with CASK. Jurkat cells were stimulated for the indicated times (0, 3, 7 or 10 minutes) with OTK3 mAb, lysed, and CASK immunoprecipitates analyzed for phosphotyrosine content with mAb 4G10 (upper panel), or for the presence of PKCθ or ZAP-70 by Western blot. Phosphorylated proteins associate with CASK after OKT3 activation, including ZAP-70 and PKCθ.

FIG. 18 summarizes the structural requirements for CASK and Cdc42/rac interaction using the depicted CASK mutants to define the minimal requirements for association with Cdc42/rac. CASK deletion constructs were co-transfected with either Cdc42/rac, RacG12V (constitutively active) or RacT17N (dominant-negative). Rac constructs were immunoprecipitated from lysates, and the presence of specific CASK constructs analyzed by Western blotting with an antibody specific to the CASK constructs. A constitutively activated mutant of Cdc42/rac (RacG12V) or a dominant-negative variant (RacT17N) exhibited no altered pattern of associations with CASK.

FIG. 19 shows results that further define the requirements for CASK binding to Cdc42/rac. Ccd42/rac was immunoprecipitated and the presence of the indicated CASK proteins monitored by Western blotting with the Au1 antibody (the numbers refer to the amino acids present in the CASK constructs). Blotting with an anti-FLAG antibody demonstrates that comparable levels of Cdc42/rac are present in each immunoprecipitate.

FIGS. 20A and 20B present binding data for Cdc42/rac and isolated domains of CASK. FIG. 20A shows results indicating that Cdc42/rac interacts with the isolated SH3-13 domains of CASK. FIG. 20B shows that the activated (RacG12V) form of Rac has no effect on binding requirements.

FIGS. 21A and 21B summarize actions of CASK on NFAT and NF-κB induction. FIG. 21A is a chart showing the opposite actions of CASK and Dlg on NFAT. Jurkat T cells were co-transfected with the indicated constructs together with a reporter plasmid that monitors T cell receptor signaling through the transcriptional activity of the nuclear factor of activated T cells (NFAT). A triplicate form of the NFAT binding site controls the expression of a luciferase reporter gene. Transfected cells were left unstimulated or stimulated with anti-CD3 antibodies, then at a later time, lysed and analyzed for luciferase activity. FIG. 21B provides results regarding NF-κB induction in Jurkat Cells. As in FIG. 21A, Jurkat cells were co-transfected with plasmids encoding CASK or Dlg in the indicated amounts in addition to a reporter construct that monitors the activity of NFκB driving a luciferase reporter gene.

FIGS. 22A and 22B concern the structure and calcium mobilization results with the CD16:7:CASK chimera. FIG. 22A is a schematic representation of the CD16:7:CASK chimeric protein consisting of the extracellular domain of CD16 and the transmembrane domain of CD7 linked to CASK. As a control, a CD16:7 chimera was constructed that lacked the membrane-linked CASK portion. FIG. 22B shows that crosslinking of the CD16:7:CASK chimera results in the mobilization of intracellular Ca+2 in Jurkat T cells. Jurkat cells expressing the indicated chimeric proteins were loaded with a calcium fluorescent dye whose fluorescence properties are altered upon binding of free intracellular calcium. Cells were stimulated with OKT3 mAb (top tracing), or anti-CD16 antibody. While engagement of the CD16:7:CASK chimera resulted in detectable mobilization of intracellular calcium (intermediate tracing), stimulation of the chimera lacking CASK sequences failed to do so (flat tracing).

FIG. 23 is a compilation of data regarding the interaction of hDlg and CASK with many proteins involved in T cell activation. It appears that CASK and hDlg bind different sets of proteins associated with lymphocyte function. Since CASK and hDlg can be co-immunoprecipitated (FIG. 16B), these molecules may associate in a macromolecular complex.

### DETAILED DESCRIPTION

### I. Definitions

As used herein, the term “PDZ domain” refers to protein sequence (i.e., modular protein domain) of approximately 90 amino acids, characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats). PDZ domains generally appear to maintain a core consensus sequence (Doyle, D. A., 1996, Cell 85: 1067-1076).

PDZ domains are found in diverse membrane-associated proteins, including members of the MAGUK family of guanylate kinase homologs, several protein phosphatases and kinases, neuronal nitric oxide synthase, and several dystrophin-associated proteins, collectively known as syntrophins. The term “PDZ domain” also encompasses variants (e.g., naturally occurring variants) of the sequence of a PDZ domain from a PDZ protein (e.g., polymorphic variants, variants with conservative substitutions, and the like). Typically, variants of a PDZ domain are substantially identical to the sequence of a PDZ domain from a PDZ protein, e.g., at least about 70%, at least about 80%, or at least about 90% amino acid residue identity when compared and aligned for maximum correspondence.

As used herein, the term “PDZ protein” refers to a naturally occurring protein containing a PDZ domain, e.g., a human protein. Exemplary PDZ proteins include CASK, hDlg1, SHANK1, SHANK3, EBP-50, KIAA0807, TIP1, PSD-95, Pick1, CNK, GRIP and DVL-2.

As used herein, the term “PDZ-domain polypeptide” refers to a polypeptide containing a PDZ domain, such as a fusion protein including a PDZ domain sequence, a naturally occurring PDZ protein, or an isolated PDZ domain peptide.

As used herein, the term “PL protein” or “PDZ Ligand protein” refers to a naturally occurring protein that forms a molecular complex with a PDZ-domain, or to a protein whose carboxy-terminus, when expressed separately from the full length protein (e.g., as a peptide fragment of 4-25 residues, e.g., 16 residues), forms such a molecular complex. Exemplary PL proteins include, but are not limited to, PAG, LPAP, ITK, DNAM-1, Shroom, PTEN, BLR-1 and fyn.

As used herein, a “PL sequence” refers to the amino acid sequence of the C-terminus of a PL protein (e.g., the C-terminal 2, 3, 4, 5, 6, 7, 8, 9, 10, 11, 12, 13, 14, 15, 16, 17, 18, 19, 20, 21, 22, 23, 24 or 25 residues) (“C-terminal PL sequence”) or to an internal sequence known to bind a PDZ domain (“internal PL sequence).

As used herein, a “PL peptide” is a peptide of having a sequence from, or based on, the sequence of the C-terminus of a PL protein.

As used herein, a “PL fusion protein” is a fusion protein that has a PL sequence as one domain, typically as the C-terminal domain of the fusion protein. An exemplary PL fusion protein is a tat-PL sequence fusion.

As used herein, the term “PL inhibitor peptide sequence” refers to a PL peptide amino acid sequence that (in the form of a peptide or PL fusion protein) inhibits the interaction between a PDZ domain polypeptide and a PL peptide.

As used herein, a “PDZ-domain encoding sequence” means a segment of a polynucleotide encoding a PDZ domain. In various embodiments, the polynucleotide is DNA, RNA, single stranded or double stranded.

A “PDZ:PL interaction” or “PDZ interaction” or “PL interaction” between a PDZ protein and a PL protein is meant to refer broadly to direct binding between these proteins though interaction with the PDZ domain of the PDZ protein.

An “interaction” between a PDZ protein and a cognate ligand protein is meant to broadly refer to direct or indirect binding between these proteins. Thus, in some instances, there is direct binding between the PDZ protein and cognate ligand protein. In other instances, the binding is indirect and is mediated by another (e.g., bridging) protein.

An “immune cell” generally refers to a hematopoietic cell, which can include leukocytes such as lymphocytes (e.g., T cells, B cells and natural killer [NK] cells), monocytes, granulocytes (e.g., neutrophils, basophils and eosinophils), macrophages, dendritic cells, megakarocytes, reticulocytes, erythrocytes and CD34+ stem cells.

The phrase “immune signaling” is meant to broadly refer a stimulation that results in a biochemical change in pathways that lead to the activation of immune cells. This activation could include, but not be limited to, phosphorylation or dephosphorylation of activation markers, cell proliferation, cytokine production, Calcium flux changes, or apoptosis.

The term “modulation” or “modulate” when used with respect to an immune signal means that a signal is inhibited or enhanced.

A “fusion protein” or “fusion polypeptide” as used herein refers to a composite protein, i.e., a single contiguous amino acid sequence, made up of two (or more) distinct, heterologous polypeptides that are not normally fused together in a single amino acid sequence. Thus, a fusion protein can include a single amino acid sequence that contains two entirely distinct amino acid sequences or two similar or identical polypeptide sequences, provided that these sequences are not found together in the same configuration in a single amino acid sequence found in nature. Fusion proteins can generally be prepared using either recombinant nucleic acid methods (i.e., as a result of transcription and translation of a recombinant gene fusion product), which fusion comprises a segment encoding a polypeptide of the invention and a segment encoding a heterologous protein, or by chemical synthesis methods well known in the art.

A “fusion protein construct” as used herein is a polynucleotide encoding a fusion protein.

As used herein, the terms “antagonist” and “inhibitor,” when used in the context of modulating a binding interaction (such as the binding of a PDZ domain sequence to a PL sequence), are used interchangeably and refer to a compound that reduces the binding of the, e.g., PL sequence (e.g., PL peptide) and the, e.g., PDZ domain sequence (e.g., PDZ protein, PDZ domain peptide).

As used herein, the terms “agonist” and “enhancer,” when used in the context of modulating a binding interaction (such as the binding of a PDZ domain sequence to a PL sequence), are used interchangeably and refer to a compound that increases the binding of the, e.g., PL sequence (e.g., PL peptide) and the, e.g., PDZ domain sequence (e.g., PDZ protein, PDZ domain peptide).

“Polypeptide” and “protein” are used interchangeably herein and include a molecular chain of amino acids linked through peptide bonds. The terms do not refer to a specific length of product. Thus, “peptides,” oligopeptides” and “proteins” are included within the definition of polypeptide. In addition, protein fragments, analogs, mutated or variant proteins, fusion proteins and the like are included within the meaning of polypeptide.

As used herein, the terms “peptide mimetic,” “peptidomimetic,” and “peptide analog” are used interchangeably and refer to a synthetic chemical compound which has substantially the same structural and/or functional characteristics of an PL inhibitory or PL binding peptide as disclosed herein. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides that are disclosed herein that are conservative variants, routine experimentation will determine whether a mimetic is a suitable mimic of the reference compound, i.e., that its structure and/or function is not substantially altered. Thus, a suitable mimetic composition is one that is capable of binding to a PDZ domain and/or inhibiting a PL-PDZ interaction. Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N=-dicyclohexylcarbodiimide (DCC) or N,N=-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN4-), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R=—N—C—N—R═) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipccolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups. A component of a natural polypeptide (e.g., a PL polypeptide or PDZ polypeptide) can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D-amino acid, but which can additionally be referred to as the R- or S-form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1992) J. Amer. Chem. Soc. 114:10672-10674. For example, a type VI beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Banerjee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421-435. See also, Hruby (1997) Biopolymers 43:219-266, Balaji, et al., U.S. Pat. No. 5,612,895.

As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are disclosed herein are partially defined in terms of amino acid residues of designated classes, the amino acids can be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids.

As used herein, the term “substantially identical” in the context of comparing amino acid sequences, means that the sequences have at least about 70%, at least about 80%, or at least about 90% amino acid residue identity when compared and aligned for maximum correspondence. An algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: -5, k-tuple=2; joining penalty=40, optimization=28; gap penalty −12, gap length penalty=−2; and width=16.

A “small molecule” typically refers to a synthetic molecule having a molecular weight of less than 2000 daltons, in other instances 800 daltons or less, and in still other instances 500 daltons or less. Such molecules can be peptide mimetics of a PDZ or PL domain, for example. Such molecules can also include segments that are polypeptides.

### II. Overview

The methods and compositions provided herein are based in part on the discovery by the present inventors that interactions between certain PDZ proteins and their cognate ligand proteins can affect the composition and/or distribution of lipid rafts in an immune cell. The inventors have examined binding interactions between a large number of PDZ and cognate ligand proteins such as PL proteins to identify those that appear to have a role in the composition and/or distribution of lipid rafts (see Tables II and III; accession numbers and pertinent references for the proteins referred to herein are provided in Table IV). Because the type of proteins present in the lipid rafts and the distribution of the lipid rafts plays a role in cell signaling, modulation of the interaction between the PDZ proteins and their cognate ligands provides a means for regulating immune cell signaling. Thus, for example, modulation of the interaction can modulate the threshold for immune cell activation. The ability to regulate immune cells in this fashion can be important in preventing an undesirable immune response or in promoting a desired immune response.

In some aspects, PDZ proteins are a group of scaffolding proteins that facilitate the assembly of multiprotein complexes, often serving as a link or bridge between proteins. The acronym PDZ reflects the names of the founding members of this class of proteins: PSD-95, Disks Large and Zona Occludens-1 (Gomperts et al., 1996, Cell 84:659-662; see also Bilder et al., 2000; Dong et al., 1997; Hata et al., 1996; Lim et al., 1999; Lue et al., 1994; Muller et al., 1995; Sheng and Sala, 2001; Staudinger et al., 1995; and Therrien et al., 1998). The PDZ family of proteins has a conserved domain of approximately 90 amino acids (i.e., the PDZ domain) that is adapted for intermolecular recognition and appears to form at least two kinds of protein-protein interactions (see, e.g., Songyang et al., 1997). One set of interactions is with the carboxy terminus (C-terminus) of cognate ligand proteins that have a basic consensus recognition motif that consists of X-T/S/Y-X-V/L/I, although subclasses of PDZ domains bind variations of this motif (see, e.g., 17 and 18, and PCT Publications WO 00/69898, WO 00/69897, and WO 0069896). PDZ domains can also interact with internal residues of some proteins, including PDZ domains themselves (see, e.g., Christopherson et al., 1999). Thus, by possessing multiple PDZ domains, PDZ proteins can act as organizers, by increasing the local concentration of one or more proteins and/or by regulating the localization of multi-protein complexes through interactions with the cytoskeleton or a specific cellular organelle. Still other PDZ proteins possess enzymatic activity and use their PDZ domain(s) to localize the enzyme with respect to its substrate. Like other modular protein interaction domains such as SH2, SH3, and WW domains, PDZ domains provide an additional means to organize or to polarize a particular complex of proteins within the cell.

Examples of PDZ proteins that the inventors have identified as having a functional role in the composition and/or distribution of lipid rafts upon binding a cognate ligand protein include hDlg (also referred to herein as hDlg1, or simply Dlg or Dlg1), SHANK1, SHANK3, EBP-50, CASK, KIAA0807, TIP1, PSD-95, Pick1, CNK, GRIP and DVL-2. The cognate ligand protein(s) to which the PDZ protein binds fall into two general classes. One class are those proteins that bind to the PDZ domain of the PDZ protein; such proteins are generally referred to herein as a “PL protein” (i.e., PDZ Ligand protein). Another class of cognate ligand proteins are those that bind to the PDZ protein at a site other than the PDZ domain. Specific examples of PL proteins which upon binding to a PDZ protein affect the composition and/or distribution of the lipid raft in an immune cell include, but are not limited to, PAG, LPAP, ITK, DNAM-1, Shroom, PTEN, BLR-1, fyn and Na+/Pi transporter.

While not intending to be bound by any particular theory, binding of a PDZ protein provided herein with its cognate ligand protein can affect the composition and/or distribution of lipid rafts in an immune cell in a number of different ways. Thus, the phrase “affect the composition and/or distribution of lipid rafts” can mean, for example, that a PDZ protein is recruited to the lipid raft (thus changing the composition of the lipid raft) by binding to a PL protein anchored in the lipid raft, or vice versa. Alternatively, a cognate ligand protein (e.g., a signal transduction protein) can bind to a region other than the PDZ domain of a PDZ protein to form an aggregate. The resulting aggregate can then become part of the lipid raft (thus changing the composition of the lipid raft) upon binding of the PDZ protein to a PL protein in the lipid raft via the PDZ domain. In yet other instances, binding of a cognate ligand protein to a PDZ protein acts to sequester the PDZ protein in the cytoplasm, thereby affecting the composition of the lipid raft.

As alluded to supra, because modulation of an interaction between a PDZ protein and a cognate ligand protein that are provided herein ultimately affects immune cell activation or deactivation, certain methods disclosed herein can be utilized to treat various immune cell disorders, including a number of autoimmune diseases, for example. A variety of screening methods are also provided. These methods are designed to identify compounds that modulate interaction between a PDZ protein and a PL protein, which proteins are disclosed herein as being able to interact with one another in an immune cell to affect the composition and/or distribution of lipid rafts.

Also provided are modulators (optionally formulated as pharmaceutical compositions) that inhibit or enhance binding between a PDZ protein and a cognate ligand protein that are disclosed herein. The modulator can be a peptide or fusion protein that comprises a certain number of residues (e.g., 2-20) from the carboxy terminus of a PL protein or a certain number of residues from the PDZ domain of a PDZ protein (e.g., 20-100). Alternatively, the modulator can be a peptide or small molecule mimetic of such peptides and fusion proteins.

### III. Interactions Between PDZ Proteins and Cognate Ligand Proteins

A. Certain PDZ Proteins that Interact with the PL Proteins PAG, LPAP and ITK

The present inventors have demonstrated that a number of PDZ proteins interact with one or more of the PL proteins called PAG, LPAP, ITK, DNAM-1, Shroom, PTEN, BLR-1, Na+/Pi cotransporter 2, and DOCK2 (see Table II for a summary of PDZ proteins that interact with PAG and LPAP). Examples of such PDZ proteins include SHANK1, SHANK3, KIAA0807, EBP-50 and TIP1. Certain of these interactions are discussed in greater detail in the following section and in the Examples infra.

1. PAG, LPAP and ITK Interactions

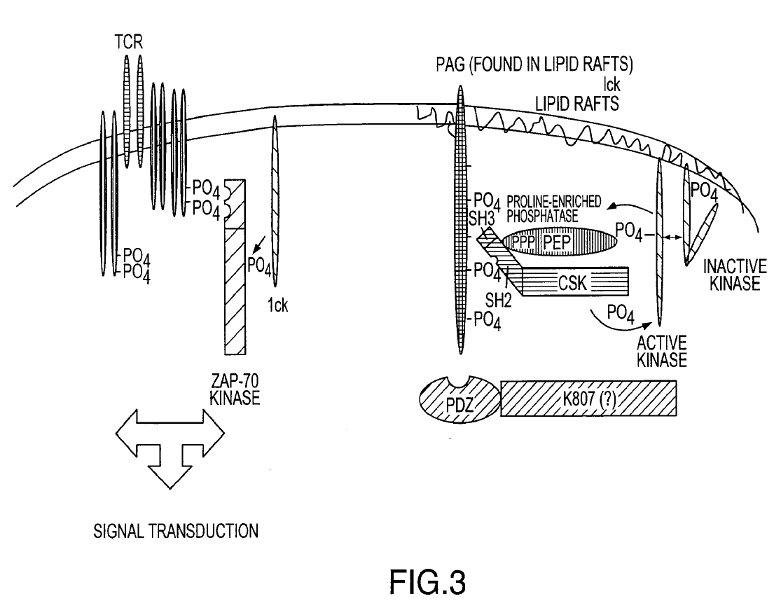

The current inventors investigated whether one or more PDZ and/or cognate ligand proteins that interact with PDZ proteins (e.g., PL proteins) were involved in regulating raft organization. One such protein that was identified is the protein PAG (phosphoprotein associated with glycosphingolipid-enriched microdomains) or CBP (csk-binding protein), which contains a PDZ-binding motif at its C-terminus. This protein is targeted to the rafts via palmitoylation and has been implicated in negatively regulating src family kinases (19, 20). As shown in FIG. 3, Src kinases, such as lck which is the kinase responsible for initiating T cell receptor signaling (20), are regulated by an intramolecular interaction between their SH2 domain and a phosphorylated tyrosine residue near the C-terminus. This interaction maintains the kinase in an inactive conformation (21). The enzyme csk (c-src kinase) is the kinase that phosphorylates this residue, thereby negatively regulating the src kinase (22). Alternatively, removal of the C-terminal phosphate activates src-kinases by allowing access of substrates to the kinase domain.

The evidence indicates that PAG inhibits src kinases by recruiting Csk to the cytoplasmic tail of PAG via a phosphotyrosine/SH2 interaction (see FIG. 3). In a resting T cell, PAG exists in its phosphorylated state, providing a docking site for csk in the raft; this places csk in close proximity with substrates such as lck. Once the T-cell antigen receptor (TCR) is stimulated, PAG becomes dephosphorylated, resulting in the release of csk from the membrane. This allows the hematopoietic-specific tyrosine phosphatase CD45 to activate lck. Overexpression of PAG in the Jurkat T cell leukemic line results in a 30-40% suppression of T cell activation, consistent with a negative role for PAG in modulating TCR signaling.

Another protein that contains a PDZ-binding motif, called LPAP (lymphocyte phosphatase-associated protein) can also regulate lck, but in an opposing fashion (23). LPAP associates with CD45, which as described supra is the phosphatase responsible for dephosphorylating the negative regulatory tyrosine residue in lck. Disruption of the LPAP gene in mice results in impaired TCR function, indicating that LPAP has a role as a positive regulator of T cell activation (24). Therefore, the PAG-csk complex likely represents a negative module, and the LPAP-CD45 complex, a positive module, with both working together to regulate the initiation of TCR signaling. Based upon these observations and results described herein, the current inventors propose that PAG and LPAP are regulated through their interaction with one or more proteins that PDZ-containing proteins, providing a means to regulate src kinase activity and thus, the threshold of T cell activation.

Since PAG is a constitutive resident of lipid rafts, by interacting with a PDZ protein it can recruit the phosphatase responsible for dephosphorylating the csk-docking site, terminating its inhibitory role. Alternatively, PAG may sequester PAG from the incoming T cell receptor within the rafts, allowing for activation to ensue. LPAP may serve as a chaperone for CD45, regulating the location of CD45 in or out of the rafts via its interaction with a PDZ domain-containing protein. Microscopy studies have shown that shortly after TCR stimulation, CD45 appears to be excluded from the immunological synapse as the lipid rafts and TCRs coalesce; at a later time, CD45 moves in and out of the synapse (25). The binding studies described herein indicate that interactions between LPAP and PDZ domains may be the mechanism by which this active shuttling occurs.

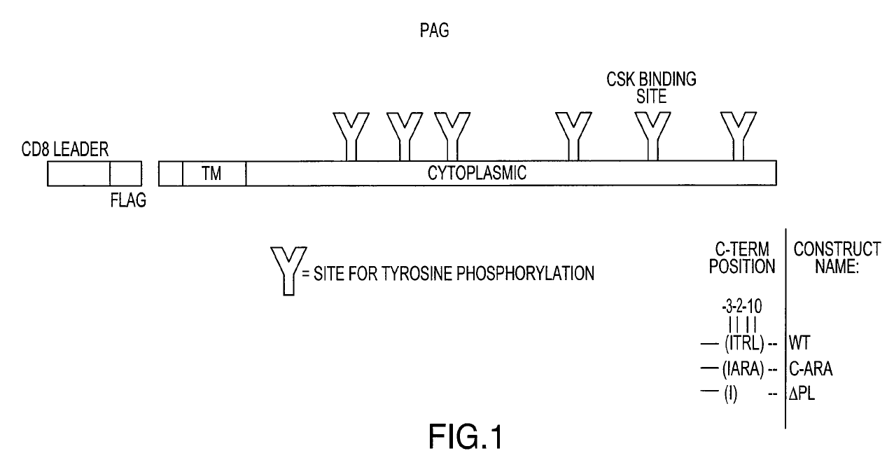

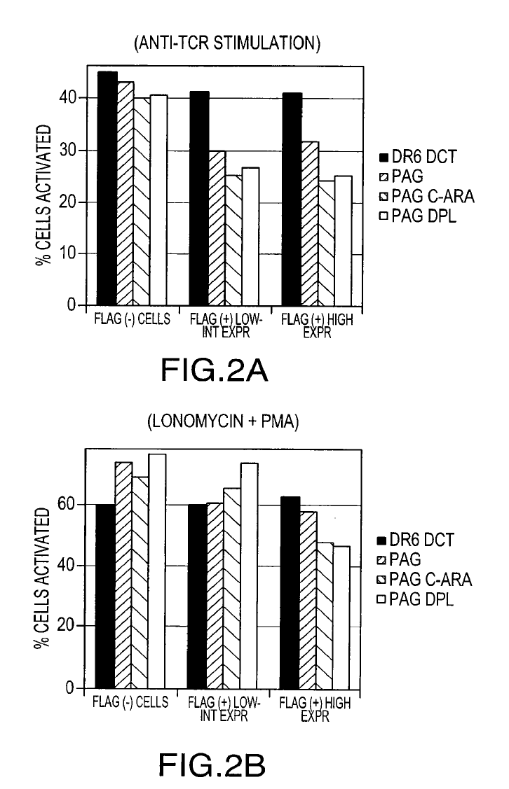

To test directly the role of the PDZ-binding motif present in PAG (ITRL), two C-terminal mutants expected to abolish PDZ binding were prepared (FIG. 1). One mutant, termed PAG C-ARA, changes the critical threonine and leucine residues whose side chains extend into the PDZ binding pocket to alanine; the second, PAG ΔPL deletes the 3 most C-terminal residues, effectively removing the PDZ ligand motif from PAG. As described in Example 1, these two mutations in the binding motif resulted in an enhanced level of inhibition; this result indicates that the PDZ interaction is important for relieving suppression by PAG on the TCR to allow for optimal activation. Thus, inhibiting the interaction between PAG and its PDZ-binding partner should decrease the sensitivity of the TCR and have a net suppressive effect on the T cell response (see FIGS. 2A and 2B).

The magnitude of the observed effect the mutations have on TCR function likely underestimates the role of the PDZ interaction for a number of reasons. First, these mutants are expressed in the presence of endogenous PAG, which still can be regulated appropriately. Second, the mutant forms still possess the capacity to bind CSK and inhibit the TCR response. Therefore, by crowding the limited area of the raft with overexpression of inhibitory PAG, the efficiency with which its inhibitory effect can be overcome is minimized. When T cells are stimulated using pharmacologic agents which bypass activation of the TCR, the suppressive effects of PAG and its mutants are seen only minimally in cells expressing the highest levels of PAG. This demonstrates that PAG works proximally in the TCR signal transduction cascade.

Another PL protein identified by the inventors as playing a role in lipid raft composition and/or distribution in lipid rafts is the TEK-family kinase ITK. ITK is recruited to the rafts upon TCR stimulation through the binding of its PH domain to the raft-localized 3,4,5 and 4,5 phosphorylated forms of phosphatidylinositol (29). In addition to it localization in the rafts, ITK binds to SLP-76 (5), an adapter protein that, together with LAT, acts to nucleate proteins that mediate mobilization of Ca+2, activation of the ras pathway, and modulation of the cytoskeleton (30). ITK has been shown to directly phosphorylate and optimally activate PLCγ1, the enzyme that produces the essential second messengers IP3 and diacylglycerol (31). Mice deficient in ITK have revealed its important contribution in thymocyte development, in determining the magnitude of the TCR-derived signal, and consequently, in the differentiation of TH2 T cells (T cells that favor an antibody-mediated immune response-see below) (32-34). Although PDZ binding by ITK is not its link to the lipid rafts, PDZ interactions may instead modulate the kinase activity of ITK, or the cohort of proteins with which it interacts, during T cell activation.

2. SHANK1 and SHANK3 Interactions

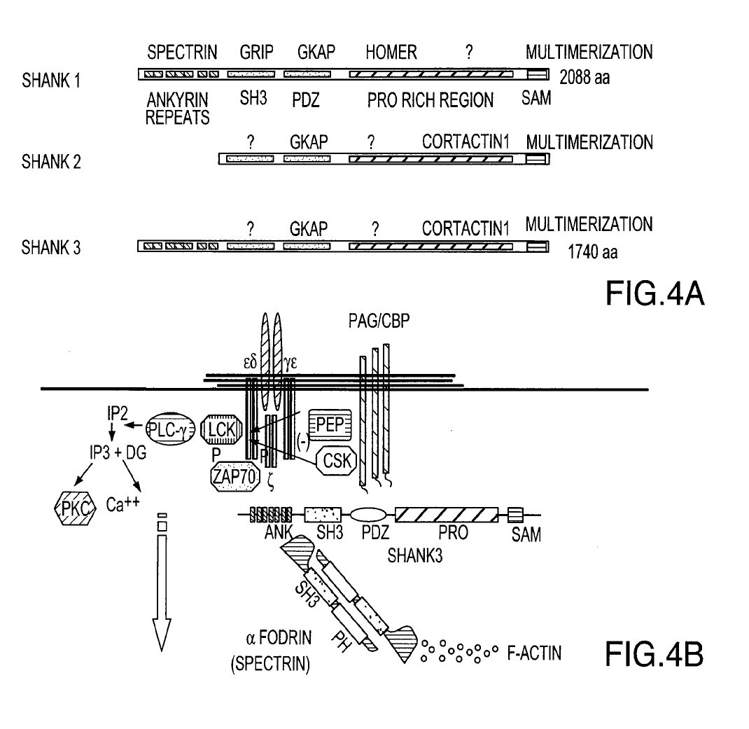

Shank proteins are a family of scaffolding proteins that only recently have been identified. They were first described as a component of the post-synaptic density in the brain (Naisbitt et al, 1999). In the rat, Shank1 and Shank3 are expressed mainly in brain, whereas Shank3 is expressed in heart, brain and spleen. As shown in FIG. 4A, Shank1, Shank2 and Shank3 contain multiple domains that act as sites for protein-protein interaction. Although the exact domains present in a particular protein varies, domains contained by the Shank proteins include N-terminal ankyrin repeats, an SH3 domain, a long proline rich region and a serial alpha motif (SAM). Shank1 interacts with the C-terminus of GKAP, a guanylate kinase-associated protein. These two proteins colocalize and mediate the interaction between PSD-95 and Shank1 in the post-synaptic density (PSD). In vitro, a Shank1 PDZ domain also interacts with the C-terminus of somatostatin receptor type 2 and metabotropic glutamate receptors.

Homer proteins, which are required for efficient signaling between metabotropic glutamate receptors and IP3 receptors (inositol phosphate receptor3), bind to the proline rich region of Shank1. Sequence similarities indicate that Shank3 also likely binds Horner. The IP3 receptors whose signaling Horner affects contain six typical membrane spanning domains in the C-terminal region that anchor the protein in the membrane. The receptor is homotetrameric and the four subunits combine to form the functional IP3-sensitive calcium channel. Once IP3 binds, it induces a conformational change that leads to the calcium channel opening.

Cortactin binding is C-terminal to Horner binding, and the evidence indicates that both Shank 1 and Shank3 bind cortactin. The serial alpha motif of the Shank proteins mediates homodimerization of Shank proteins, allowing them to multimerize tail to tail. In rats, Shank2 and Shank 3 bind to the SH3 domain of cortactin, an actin-interacting protein that links Shank to the cytoskeleton in post-synaptic densities. The SH3 domain of Shank 1 binds to GRIP (glutamate receptor interacting protein), a 120 kD protein found in the postsynaptic terminal that contains 7 PDZ domains.

As shown in FIG. 4B, in brain extracts and transfected cells, the N-terminal ankyrin repeats of Shank1 and Shank3 interact with alpha fodrin or spectrin, an actin binding protein composed of two chains, an alpha chain that binds to ankyrin repeats and the beta chain that binds actin protein. The fact that Shank proteins interact with alpha fodrin/spectrin indicates that Shank proteins serve in various structural roles, since components of the cortical cytoskeleton like ankyrin and spectrin are also associated with cross-linked CD3. In addition, spectrin is involved in the capping of T and B cells after antibody cross-linking of lymphocyte receptors.

As described in greater detail in Example 4, the current inventors have now identified the protein PAG as a ligand for Shank1 and Shank3 PDZ domains (see FIG. 4B). As described supra, PAG or Cbp is a Csk-binding protein in the brain (Kawabuchi M. et al. 2000) and is a phospho-protein associated with lipid rafts in lymphocytes (20). The binding between PAG and Csk means that PAG has a role in controlling immune response because, as set forth above, Csk is involved in the negative regulation of T-cell immune responses by phosphorylating the C-terminus of the src kinases Lck and Fyn, thus inactivating them. The results provided herein show that binding between PAG and Csk increases this kinase activity of Csk and that Csk binds to phosphorylated PAG/Cbp through its SH2 domain and is recruited to lipid rafts.

Thus, collectively, the results indicate that the PAG/Shank3 complex serves as a bridge between the lipid rafts containing the signaling machinery associated with the TCR and the cytoskeleton, and that this complex is involved in the formation and reorganization of the immune synapse (see FIG. 4B). More specifically, as described supra and illustrated in FIG. 3, in resting T cells PAG is phosphorylated and binds csk via an SH2 domain of csk, with csk further binding proline-enriched phosphatase (PEP). Binding of csk to PAG positions PAG to phosphorylate lck, thus inactivating it. When the T cell is activated, however, PAG becomes dephosphorylated which results in the release of csk. This release allows a phosphatase to approach lck and dephosphorylate it, thereby activating lck to initiate activation of the T cell.

3. KIAA0807 Interactions

In ELISA-based assays described in Example 4, the inventors demonstrated that the protein encoded by the KIAA0807 gene (Genbank Accession No. 3882334) can bind to the C-terminus of both PAG and LPAP. The KIAA0807 gene encodes a protein that contains a single PDZ domain followed by a region that exhibits high degree of homology to a kinase domain. Since phosphorylation of a PL motif can change its binding specificity (35), the proximity of a kinase to the KIAA0807 PDZ domain may help determine whether PAG or LPAP is bound at any given time. KIAA0807 protein may reside outside the raft and therefore, be responsible for sequestering PAG from the TCR following activation. It may also mediate the exclusion of the LPAP/CD45 complex from the raft that is observed shortly after TCR engagement. Alternatively, KIAA0807 protein may be bound to PAG in the basal state, preventing PAG from binding the phosphatase that inactivates PAG through dephosphorylation of the csk-binding site. Hence, selective interruption of KIAA0807 binding to either LPAP or PAG, e.g., with a PL mimetic, can be used to alter the immunoreceptor signaling threshold.

4. TIP1 Interactions

The inventors have also shown that TIP1 (38), a protein consisting of a single PDZ domain and virtually nothing else, can bind to the C-terminus of LPAP (see Example 4 and Table II). While a protein of this configuration would not be expected to organize protein complexes or control cellular localization, it could act as a competitor, preventing LPAP from binding to another partner such as hDLG (see infra) or KIAA0807. Alternating binding of LPAP to hDlg, KIAA0807 or TIP1 could account for the movement of LPAP/CD45 into and out of the rafts following TCR engagement.

B. Interactions Between the PDZ Proteins hDlg1 and CASK with Cognate Ligand Binding Proteins

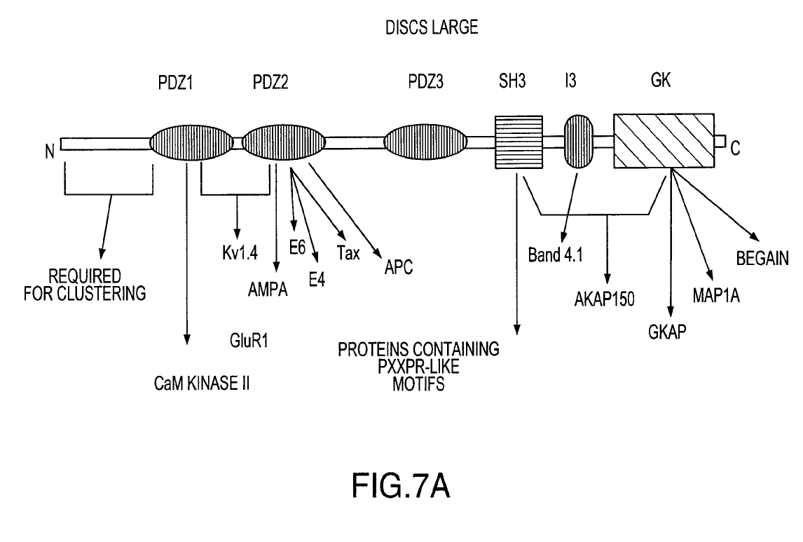

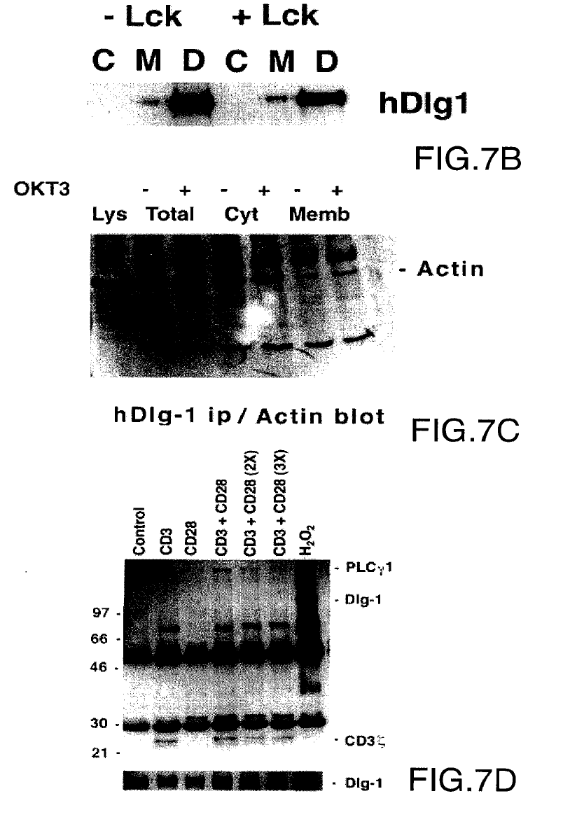

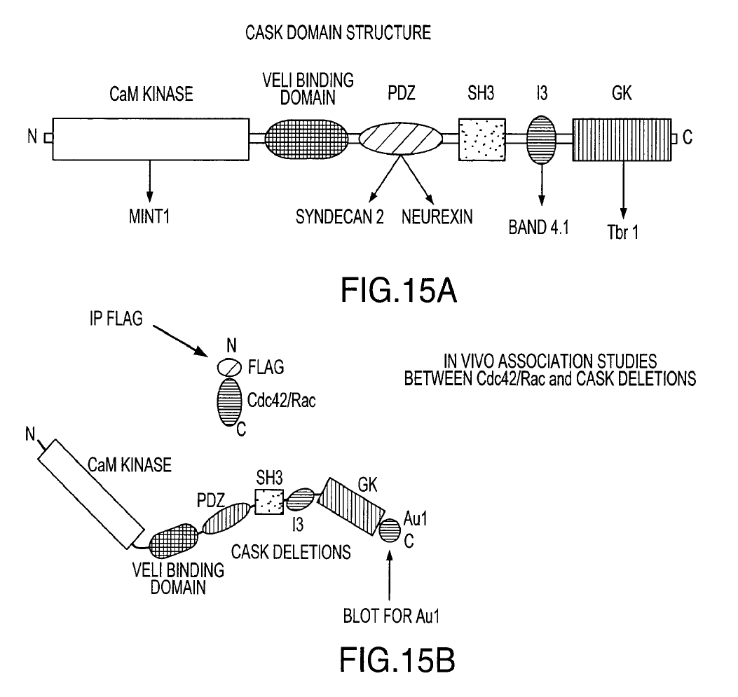

The current inventors have also demonstrated that certain PDZ proteins partition T cell signaling molecules into distinct subgroups that reflect anatomical and functional divisions of the antigen response. One subset, associated with the human homolog of Drosophila Discs Large, hDlg1 (also referred to herein as hDlg, Dlg1 or Dlg), appears to contain the early participants in the signaling process and can lead to cell death and signaling extinction if chronically engaged. FIG. 7A presents a schematic representation of hDlg and summarizes some of the proteins that interact with the various domains. Another subset, associated with CASK, contains many of the molecules that are associated with induction of transcriptional activation events (see FIG. 15A).

1. Associations Involving hDlg1 and CASK in Lipid Rafts of T-Cells

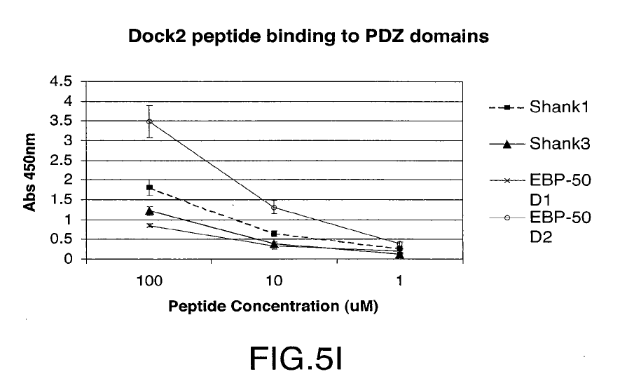

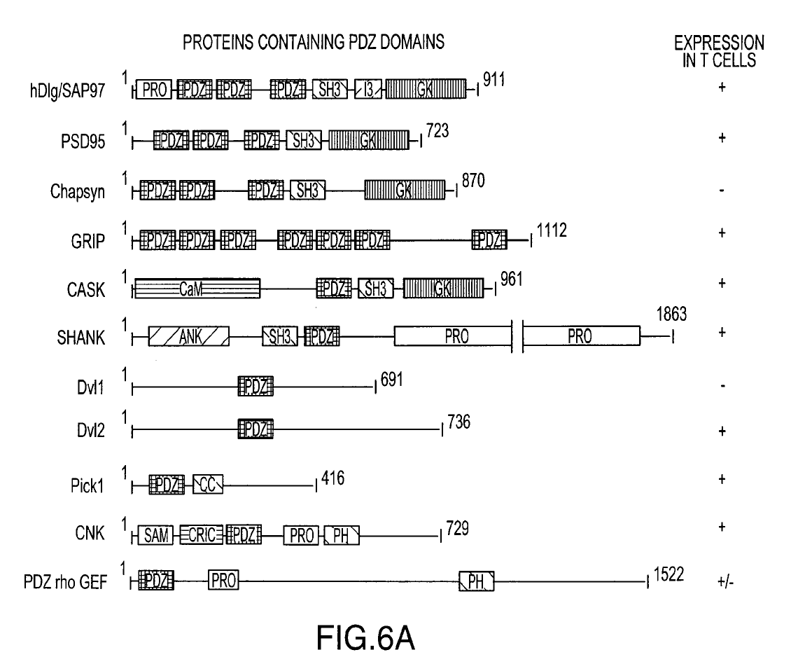

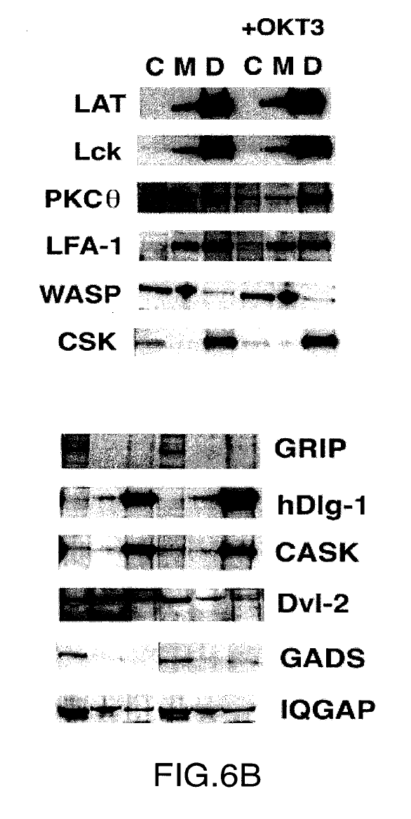

An initial set of immunoblot experiments (see Example 5) was performed to identify PDZ proteins in the Jurkat cell line and to examine association with membranes lipid rafts. (FIG. 6A). FIG. 6B shows that the PDZ proteins hDlg1, CASK, PSD-95, GRIP, Shank, Dvl-2, Pick1 and CNK are present in human T cells, and that hDlg1 and CASK associate with lipid rafts, whereas Dvl-2 and GRIP are not significantly enriched in these microdomain fractions. FIG. 6B also shows that LFA-1 is equally represented in lipid rafts and the bulk membrane, whereas the concentration of PKC-θ and GADS in the microdomain fraction increases significantly during activation induced by treatment of cells with the monoclonal antibody OKT3 (Bi et al., 2001). As shown in FIG. 6B, WASP and IQGAP, proteins implicated in actin filament interaction and reorganization, are represented predominantly in the cytosolic and membrane fractions. In T cells, hDlg1 has been shown to form a stable complex with the Src family kinase Lck, which is constitutively present in microdomains, and to associate with band 4.1 protein, a component of the membrane skeleton (Hanada et al., 1997; Hanada et al., 2000). However, FIG. 7B shows that hDlg1 remains associated with lipid rafts in cell lines that lack Lck, indicating that some other mechanism guides hDlg1 to the membrane lipid rafts.

2. Dlg1 Associates with Membrane Actin Cytoskeleton on TCR Activation

Among the PDZ proteins that are enriched in membrane microdomains, hDlg1 and CASK are structurally distinguished by a medial i3 domain that is thought to interact with ezrin-radixin-moesin family proteins, which serve to couple membrane proteins to the actin skeleton (Thomas et al., 2000; Wu et al., 1998). To assess the effect of TCR activation in regulation of actin association, hDlg1 was immunoprecipitated from the cytosolic and membrane fractions of Jurkat T cells that had been exposed to agonistic antibody (anti-CD3, specifically OKT3) stimulation. As shown in FIG. 7C, hDlg1 in the cytosolic fraction constitutively associates with actin, whereas hDlg1 from the membrane fraction undergoes an activation-dependent increase in association with actin upon stimulation. Although CASK contains a similar i3 domain, it does not associate with membrane actin, either basally or upon activation, but interacts with cytosolic actin (see Example 14).

To better understand the morphological consequences of Dlg1 and CASK interactions with actin, 293T cells and Jurkat cells transfected with green fluorescent protein (GFP) tagged fusion proteins were examined by photomicroscopy (see Example 10). The rat homologue of Dlg1 colocalizes with cortical actin cytoskeleton, whereas CASK is predominantly cytosolic. Antibody-mediated patching of the TCR under conditions that favor microspike formation leads to an increase in Dlg1-cortical actin association, with overlap seen in microspikes protruding from the Dlg1-GFP transfected cells. To analyze the effects of receptor-ligand interactions, Dlg1-GFP or CASK-GFP transfected Jurkat cells were co-cultured with an equal number of Raji B cells in the presence of the superantigen staphylococcal enterotoxin D (SED) (Fraser et al., 1992; Shapiro et al., 1998). Actin colocalized with Dlg1 on activation, whereas CASK and actin colocalization at the contact interface did not reach statistical significance. T cell-B cell conjugates formed in the absence of superantigen failed to accumulate actin at the T cell-B cell contact interface.

3. Association Between hDlg and Signaling Molecules

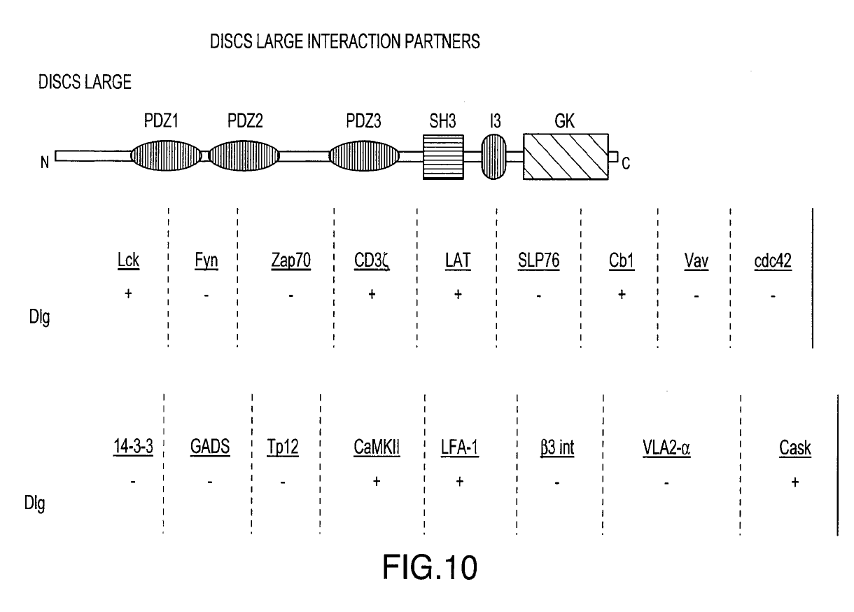

As discussed supra, in T cells hDlg forms a stable complex with the Src family kinase, Lck, which is constitutively present in membrane microdomains. To identify other T cell signaling molecules that coassociate with hDlg1, and to explore the possible effects of T cell activation on their association, endogenous hDlg1 from Jurkat T cells was immunoprecipitated and the resulting immunoprecipitates analyzed for the presence of various molecules by immunoblot analyses. FIGS. 7D, 9 and 10 show that, in addition to Lck, the signaling molecules Cbl, LAT, PLCγ1 and CD3ζ are associated with hDlg1 in the resting state (see Table III), as is the integrin LFA-1 (CD11a/CD11b). However the related proteins SLP76, GADS, and a number of other partners of the above molecules (e.g., CD45, Cdc42, Fyn, ZAP-70, VLA2α, Tp12, β3 int, and 14-3-3; see Table III) are not found in complexes with hDlg1 (FIG. 10). Upon activation, the relative amounts of LFA-1 and CD3ζ coordinated by hDlg1 increase, whereas the amounts of Vav1 decrease. Immunoprecipitations with isotype controls for each experiment were performed. The CD3ζ complexed with hDlg1 contains both phosphorylated and nonphosphorylated species, and the phosphorylated form is detected in wild-type and ZAP-70-deficient cells, but not in Lck-deficient cells.

4. Endogenous CASK Interacts with CD3ζ and Cytosolic Adaptor Molecules in T Lymphocytes

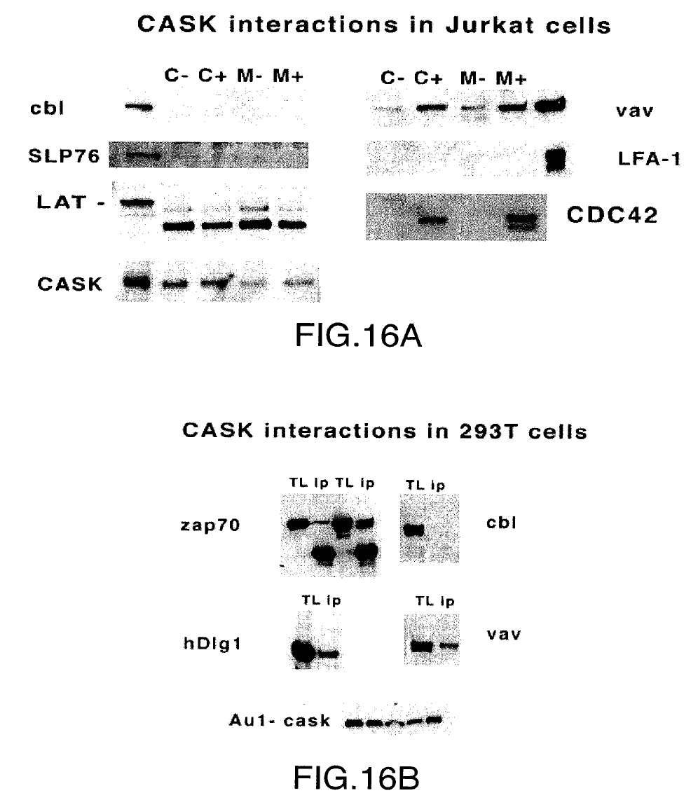

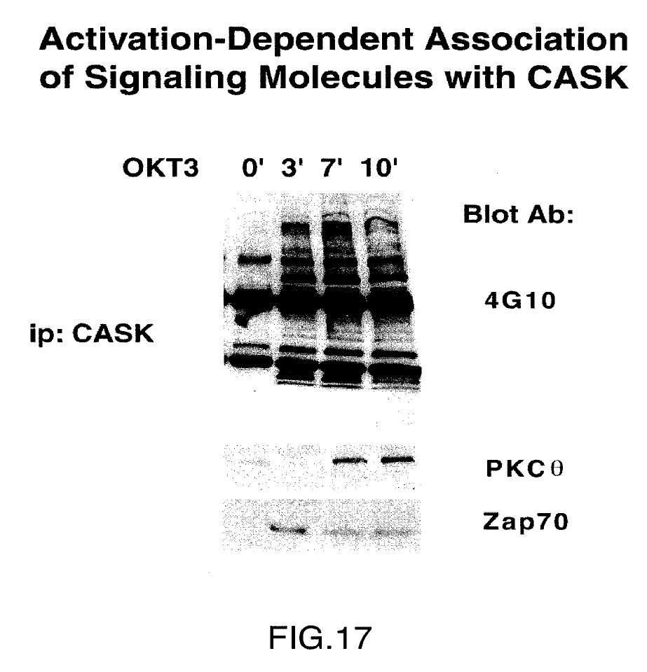

As with hDlg, immunoprecipitation experiments were conducted to identify molecules that are associated with CASK (see, for instance, Examples 13-14). Although CASK contains a similar i3 domain, it differs from hDlg in that it has an extra N-terminal region consisting of a CaM kinase like domain (see FIG. 15A). Immunoprecipitation of endogenous CASK from Jurkat cells shows that unlike hDlg1, CASK does not form complexes with LAT or LFA-1, but instead has the ability to associate with Vav1, Cdc42, ZAP-70 and hDLG (FIGS. 16A and 16B). The affiliation with the latter molecules shows a different pattern upon activation, however, as treatment with agonistic antibodies leads to a marked increase in the associations with Vav1 and PKC-ζ (FIG. 17). Unlike hDlg1, CASK interacts with ZAP-70, and the interaction increases upon activation (FIG. 17). Isotype controls for each antibody were conducted. Experiments were also conducted to determine if monomeric G proteins interact with CASK and Dlg1 on T cell activation. CASK bound to the small monomeric G proteins such as Ras. Ras interaction with CASK complexes is temporally regulated, peaking at 5 minutes following exposure to agonistic antibodies.

5. Multiple T cell Signaling Molecule Immunoprecipitates in T cells Differentially Associate with Scaffold Proteins hDlg and CASK

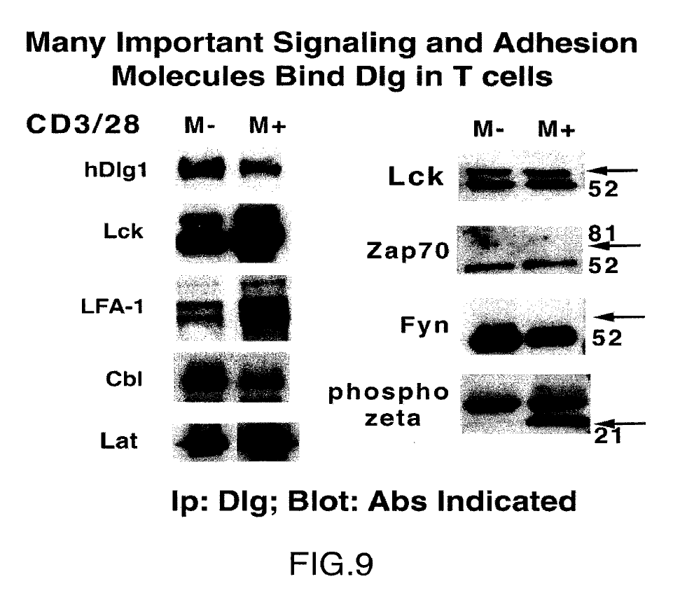

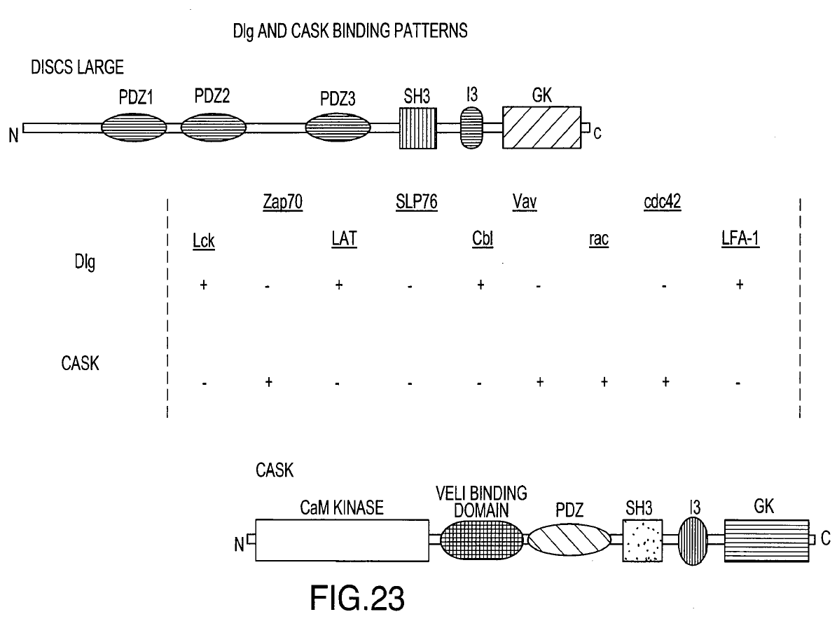

Communoprecipitation experiments were performed to examine the interactions of various signaling molecules with the PDZ domain containing proteins hDlg and CASK. The results shown in FIGS. 9 and 10 show that hDlg associates with Lck, CD3ζ, LAT, Cbl, CaMKII, LFA-1 and CASK. FIG. 16 shows that CASK, on the other hand, can associate with Vav, Cdc42, ZAP-70 and hDlg, whereas hDlg did not show association with Vav, Cdc42, and ZAP-70 (FIGS. 9 and 10). These results indicate that hDlg and CASK organize different sets of proteins involved in lymphocyte activation (summarized in FIG. 23) and can bring them together since they themselves self-associate.

6. Dlg and CASK Interactions with T Cell Signaling Molecules can be Reconstituted in Heterologous 293 Cells

Studies were then conducted to evaluate whether the interactions detected in Jurkat cells could be documented in nonlymphoid cells as well. Such experiments were conducted by expressing hDlg and candidate interacting proteins in human embryonic kidney 293 cells. Specific associations between hDlg and CD3ζ, LAT, lck, cbl, CASK LFA-1 and CaMKII were documented in 293 cells; whereas, associations with ZAP-70, fyn, SLP-76, vav, Cdc42, GADS, Tp12, β3 integrin, VLA2-α and 14-3-3 were not apparent in the absence of the other constituents (FIG. 10). Simple deletion or point mutation studies showed that the association with CD3ζ and lck depended on the N-terminal region of Dlg (data not shown).

It was found that tagged forms of hDlg1 and CASK associate with CD3ζ chain when constructs encoding the scaffold proteins are cotransfected in 293 cells with a construct encoding a chimeric CD4:ζ fusion (Romeo, 1991). Association of Vav-1 with CASK but not hDlg1 can also be shown under these conditions (FIG. 16B). Another set of experiments were conducted to determine if the interaction between Ras and CASK could be documented in 293T cells following transient transfection of Au1 tagged CASK with wild type Ras or constitutively active forms of Ras. CASK binds well to various other forms of activated Ras, e.g., RasG12VY40C, Ras G12VT35S and RasG12VE37G. In parallel experiments, Dlg1 binds to neither wild type nor mutationally activated Ras (e.g., Ras G12V). Similarly, the Cbl:hDlg1 interaction and the monomeric G protein:CASK interaction are preserved in 293 cells. Preliminary mapping experiments showed that the Cbl:hDlg1 association requires the distal portion of hDlg1, whereas the G protein Cdc42:CASK association requires sequences between residues 337 and 600. As with other attempts to map protein-protein interactions on scaffold proteins, identification of specific domain associations can be complicated by multivalent interaction, and several examples of polyvalent positive and negative contributions have been found.

7. Superantigen Induced T Cell-B Cell Complexes Differentially Recruit hDlg and CASK

In order to identify morphological correlates to biochemical interactions identified in T cells, experiments analyzing co-localization of CASK and hDlg following T cell-B cell conjugation in the presence of superantigen were conducted. Dlg1-GFP or CASK-GFP transfected Jurkat cells were co-cultured with an equal number of Raji B cells in the presence of the superantigen staphylococcal enterotoxin D (SED) (Fraser et al., 1992; and Shapiro et al., 1998). The results indicate that although there is considerably more Dlg1 than LFA-1, LFA-1 colocalizes with membrane Dlg1, whereas the CASK expression pattern overlaps with that of Vav1 and of activated PKC-θ (detected with a phospho-PKC-θ-specific antibody) at the conjugate interface (data not shown). Reciprocal staining and overlap microscopy experiments confirm several of the key features identified by biochemical analysis. Vav1 association with Dlg1 appears to be retained in the superantigen/microscopy system, whereas it diminishes with time in the agonistic antibody/immunoprecipitation system.

8. hDlg Overexpression Activates Annexin Positive T Cell Apoptosis

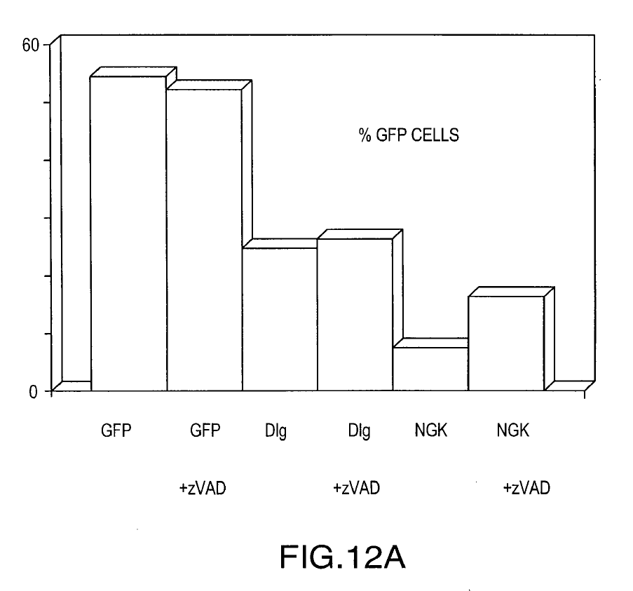

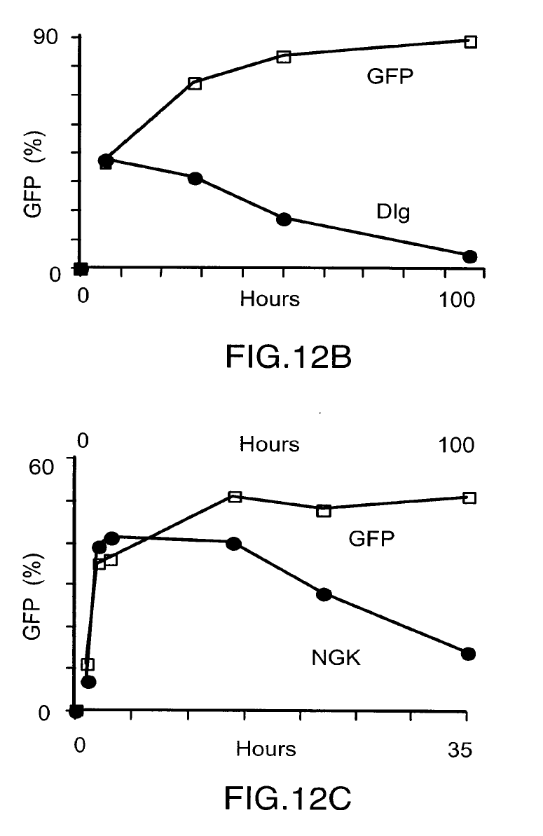

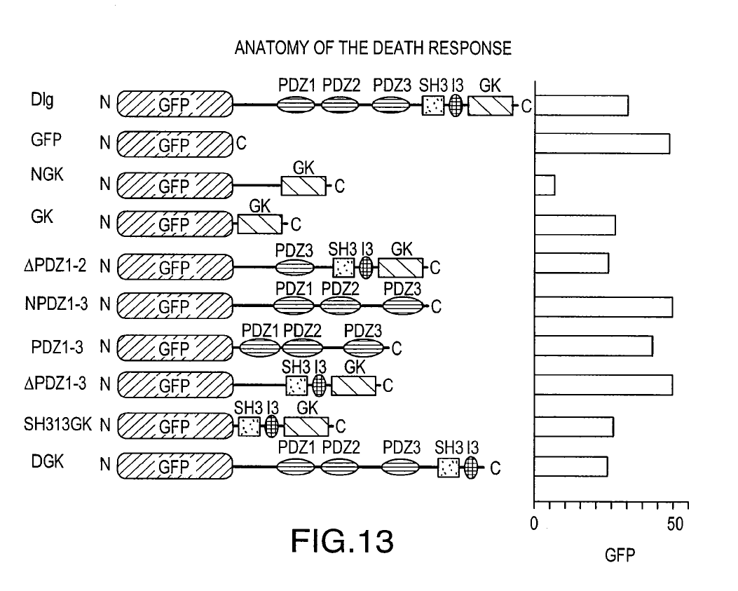

In other systems, the study of the contributions of scaffolding proteins has been difficult to assess precisely, possibly because of the plethora of binding interactions and the likelihood that substantial functional redundancy among the proteins as a group frustrates the identification of specific circuits. In T cells, overexpression of these molecules results in a significant induction of cell death (FIGS. 12A-C and FIG. 13) that has many of the characteristics of apoptosis, including outer leaflet display of phosphatidylserine (Annexin V reactivity) and chromatin fragmentation (TUNEL assay, not shown). FIG. 13 also shows that hDlg itself, or an internally deleted version of hDlg retaining the N-terminal domain and the guanylate kinase domain (Dlg1NGK) are cytotoxic. The N-terminal domain may be required for toxicity because it bears determinants responsible for localizing the molecule, whereas the C-terminal domain may be directly responsible for effector function. When expressed in human embryonic kidney 293T cells, the GFP constructs encoding Dlg1-GFP, Dlg1NGK-GFP, and GFP produced comparable levels of fluorescence.

9. Scaffold Proteins Differentially Activate NFAT and NF-kB on T Cells Activation

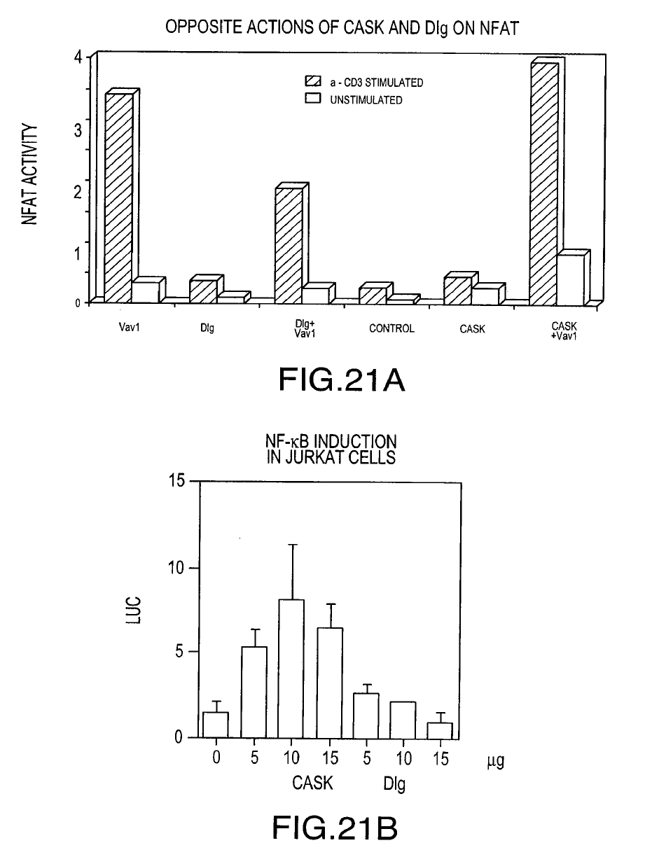

In Jurkat cells that have been partially protected against cell death by coexpression of antiapoptotic proteins, overexpression of hDlg or CASK has dissimilar consequences. Overexpression of CASK leads to basal activation of NF-κB (FIG. 21B), and a distal segment encompassing the guanylate kinase domain slightly antagonizes basal NF-κB activity (data not shown). In contrast, intact Dlg1 antagonizes basal activity and inhibits the induction due to cotransfected Vav1 (FIG. 21B). A carboxy-terminal fragment of Dlg1 modestly synergizes with Vav1 to give higher basal NF-κB activity. CASK activates NFAT modestly and in this context, the carboxyl terminal domain has full activating potential. Dlg1 inhibits Vav1-induced basal and CD3-potentiated NFAT activity and both an amino terminal and a carboxy terminal fragment act in the opposite sense to the intact molecule (FIG. 21A). Together these data suggest that Dlg1 may play a role in attenuating receptor-dependent activation, whereas CASK may be involved in coordinating molecules that lead to activation and the engagement of the transcriptional machinery. The former role may be consistent with the initial identification of Dlg1 as an inhibitor of cellular proliferation.

10. Summary of Interactions Involving Dlg1 and CASK

The PDZ proteins examined affiliate with lipid rafts and the pattern of their associations appears to partition many of the most important signaling molecules into discrete and largely nonoverlapping sets. A number of the molecules coordinated by these scaffold proteins lack the characteristic C-terminal motifs associated with PDZ domain binding. Preliminary mapping studies indicate that different parts of the scaffolds are required for interaction with certain client proteins and may correlate with the different temporal patterns of association and dissociation. Upon activation, the hDlg1 complex contains increased amounts of LFA-1, CD3ζ and actin, and decreased amounts of Vav1. The CASK complexes, in contrast, show increased amounts of Vav1 and PKC-θ, as well as CD3ζ and ZAP-70. Activated G proteins affiliate with the CASK complexes, indicating that these complexes contain many of the principal transducers of early T cell activation.

### IV. Modulating Immune Cell Signaling

A. Methods

Immune cell (e.g., T cells or B cells) antigen recognition is associated with the formation of a structured interface between antigen-presenting and responding cells which facilitates transmission of activating and desensitizing stimuli. As described in the preceding sections, proteins that include PDZ domains organize signaling molecules into discrete supramolecular complexes with distinct properties. Thus, for example, an interaction between a PDZ protein and a cognate ligand protein such as a PL protein can affect the composition and/or distribution of lipid rafts in an immune cell and, in so doing, can control the threshold at which an immune cell is activated or deactivated.

These findings can be utilized in methods to treat patients suffering from a number of immune disorders. In general such methods involve modulating an interaction between a PDZ protein and a cognate ligand protein, such modulation influencing the constituents and organization of the lipid rafts to inhibit or promote a particular immune cell signal. The modulation can involve modulating an interaction between any of the PDZ proteins and corresponding cognate ligand protein disclosed herein (see, e.g., Tables II and III). In some instances, the interaction that is modulated is one between the PDZ domain of a PDZ protein and carboxy terminal residues of a PL protein. In other instances, the interaction is between a PDZ protein and a cognate ligand protein that interacts with the PDZ protein at a domain other than the PDZ domain.

Thus, for example, by modulating the interaction between a PDZ protein such as hDlg, SHANK1, SHANK3, EBP-50, CASK, KIAA0807, TIP1, PSD-95, Pick1, CNK, GRIP and DVL-2 with a cognate ligand protein, one can modulate the threshold of immune-receptor function. Similarly, by modulating the interaction between PL proteins such as PAG, LPAP, ITK, DNAM-1, Shroom, PTEN, BLR-1 and fyn, for example, one can also modulate immune cell activation and deactivation. As a more specific example, one can modulate the function of CD45 in B and T cells by modulating the interaction between a PDZ protein and LPAP. In a related fashion, the activity of receptors that utilize the src-family of kinases in their signaling cascades can be modulated by altering the interaction between a PDZ protein and PAG, for instance.

Some methods for modulating immune cell function involve administering a compound that inhibits or enhances interaction between one or more of the PDZ proteins and a cognate ligand protein (e.g., a PL protein) which are disclosed herein. The amount of compound administered to the patient is a therapeutically effective or prophylactically effective amount. A “therapeutically effective” amount is an amount that is sufficient to remedy a disease state or symptoms, particularly symptoms associated with immune disorders, or otherwise prevent, hinder, retard, or reverse the progression of disease or any other undesirable symptoms in any way whatsoever. A “prophylactically effective” amount refers to an amount administered to an individual susceptible to or otherwise at risk of a particular disease to prevent, retard or lessen the progression of the disease or the undesirable symptoms associated with the disease. The compound can be an agonist or antagonist of the interaction between the PDZ protein and the cognate ligand protein. As described infra, such compounds can include, for example, at least a portion of the residues (e.g., 2-20 residues) from the carboxyl terminus of a PL protein or from the PDZ domain of a PDZ protein. Alternatively, the compound can be a polypeptide or small molecule mimetic of such compounds.

The methods can be utilized to treat disorders associated with improper immune signaling, such as a number of autoimmune diseases and non-autoimmune diseases. Autoimmune diseases arise when potentially autoreactive T cells that are normally refractory, become sensitized to respond against the host cells. Therefore, increasing the threshold required for T cell activation can ameliorate many autoimmune diseases and, in addition, can be utilized to reduce transplantation rejection. Alternatively, sensitizing T or B cell reactivity can enhance an immune response that is insufficiently strong to fight a particular pathogen, virus, or tumor. Evidence shows that the magnitude of the TCR signal can dictate the polarity of the immune response, i.e., whether or not the response is predominantly a cellular (TH1) or antibody-mediated (TH2) response (39, 40). Many autoimmune diseases are characterized by populations of T cells that are skewed in their differentiation profile as defined by the cytokines they produce. TH1 cells are predominantly biased towards the production of IL-2 and γ-interferon, while TH2 cells secrete predominantly IL-4, IL-5, IL-10, and IL-13. Some pathogens are effectively cleared by one type of response but not the other (41). By diminishing or enhancing the TCR signal, the potential exists to change the polarity of the immune response from a deleterious to a beneficial one. As mentioned above, T cells deficient in the PL-containing kinase ITK, are impaired in mounting TH2 responses and instead, are biased towards predominantly TH1 immunity (34); therefore, ITK and its PDZ ligand would likely be a good target for modulating the TH1/TH2 profile of T cells during an immune response.

Concerning the PL motif in LPAP and PAG as targets, while the function of LPAP in regulating CD45 is restricted to immune cells, PAG is ubiquitously expressed. Therefore, modulating activity of PAG would have the capacity to regulate all receptors that utilize src kinases, such as those regulating mast cell degranulation, platelet activation, bone metabolism, and growth factor responses to name only a few.

Exemplary diseases that can be treated according to the methods provided herein include, but are not limited to, systemic lupus erythematosus (SLE), multiple sclerosis, diabetes mellitus, rheumatoid arthritis, inflammatory bowel syndrome, psoriasis, scleroderma, inflammatory myopathies, autoimmune hemolytic anemia, Graves disease, Wiskott-Aldrich syndrome, lymphoma, leukemia, severe combined immunodeficiency syndrome (SCID) and acquired immunodeficiency syndrome (AIDS).

### V. Modulators of Immune Response

A. Chemical Characteristics

In view of the binding information between PDZ proteins and cognate ligand proteins (e.g., PL proteins) that is provided herein, agonists and antagonists of such interactions can be synthesized or identified from libraries utilizing any of a number of screening methods, including those described infra. Certain of these compounds can then be utilized in the treatment methods described in the preceding section.

Some modulators of the interactions set forth herein, particularly inhibitors, can be designed based upon the motifs of the PDZ and cognate ligand proteins that interact with one another. Based on the disclosure herein, it will be within the ability of the ordinary practitioner to identify modulators of specified PDZ-PL interactions using standard assays (see, e.g., infra). For instance, certain antagonists have a structure (e.g., peptide sequence or peptide mimetic structure) based on the C-terminal residues of PL-domain proteins. Other antagonists have a structure that mimics the residues located in the PDZ domain of a PDZ protein disclosed herein as functioning in immune cell signaling. Thus, for instance, such antagonists are designed to have a structure that includes (or mimics) 2 to 20, or 30, or 40 residues (including any integral number of residues therebetween) from the C-terminus of a PL protein disclosed herein. Other antagonists are designed to include (or mimic) 2 to 100 residues (or any integral number of residues therebetween) from the PDZ domain of a PDZ protein disclosed herein. If a cognate ligand protein is a protein other than a PL protein, then the antagonist can be designed to mimic the particular motifs involved in the interaction between the particular PDZ protein and cognate ligand protein. Certain modulators are fusion proteins that include residues from the PDZ or PL domains in addition to another polypeptide moiety.

Other compounds, including antagonists as well as agonists, have structures that are not based upon the motifs involved in the interaction. Compounds having the desired activity can readily be identified according to the screening methods discussed infra

The compounds that act as modulators can have widely varying chemical composition. For instance, certain compounds are polypeptides; other compounds are small molecules prepared by synthetic chemical methods that are mimetics of motifs involved in a particular interaction of interest. Some of these compounds are tetrazole-based compounds. Such compounds can be useful because tetrazoles resemble the C terminus of polypeptides but are able to cross cell membranes more readily. Other compounds can be β-lactams, heterocyclic compounds, oligo-N-substituted glycines, and polycarbamates, for example.

B. Formulation of Modulators as Pharmaceutical Compositions

1. Composition/Formulation

One or more of the agonists or antagonists disclosed herein can be combined with a pharmaceutically acceptable carrier as part of a formulation or medicament for use in treating various immune related diseases, such as those described supra. The compositions can also include various compounds to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant, for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. Polypeptides can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

2. Dosage

The pharmaceutical compositions can be administered as part of a prophylactic and/or therapeutic treatments. As indicated supra, a “therapeutically effective” amount refers to an amount that is sufficient to remedy a disease state or symptoms, particularly symptoms associated with immune disorders, or otherwise prevent, hinder, retard, or reverse the progression of disease or any other undesirable symptoms in any way whatsoever. A “prophylactically effective” amount refers to an amount administered to an individual susceptible to or otherwise at risk of a particular disease to prevent, retard or lessen the progression of the disease or the undesirable symptoms associated with the disease.

Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred.

The data obtained from cell culture and/or animal studies can be used in formulating a range of dosages for humans. More specifically, the effective doses as determined in cell culture and/or animal studies can be extrapolated to determine doses in other species, such as humans for example. The dosage of the active ingredient typically lines within a range of circulating concentrations that include the ED50 with little or no toxicity. The dosage can vary within this range depending upon the dosage form employed and the route of administration utilized. What constitutes an effective dose also depends upon the nature of the disease and on the general state of an individual's health.

3. Administration

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules which consist of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### VI. Screening Methods

With knowledge of the PDZ interactions disclosed herein, one can identify modulators of a particular PDZ/cognate ligand protein (e.g., PL protein) interaction according to a number of different screening methods. For example, in certain assays, a test compound can be identified as an modulator of binding between a PDZ protein and a cognate ligand protein (e.g., a PL protein) by contacting a PDZ domain-containing polypeptide and a polypeptide having a sequence of a PDZ ligand (e.g., a peptide having the sequence of a C-terminus of a PL polypeptide) in the presence and absence of the test compound, under conditions in which they would (but for the presence of the test compound) form a complex, and detecting the formation of the complex in the presence and absence of the test compound. It will be appreciated that less complex formation in the presence of the test compound than in the absence of the compound indicates that the test compound is an inhibitor of a PDZ protein-PL protein binding and greater complex formation is indicative that a compound enhances binding. Such modulators (whether found by this assay or a different assay) are useful to modulate immune function.

Certain of the current inventors have described in considerable detail assays that can be utilized to screen for compounds that modulate (e.g., inhibit) interactions between PDZ proteins and their cognate ligand proteins (see, e.g., the “A” and “G” assays described in PCT Publications WO 00/69896, WO 00/69898 and WO 00/69897). In general, these methods involve immobilizing either a PL protein or PDZ protein (or at least domains therefrom) to a surface and then detecting binding of a PDZ or PL protein (or fusion proteins containing domains thereof), respectively, to the immobilized polypeptide in the presence or absence of a test compound.

Generally, assay methods such as just described are conducted to determine if there is a statistically significant difference in the amount of complex formed in the presence of the compound as compared to the absence of the test compound. The difference can be based upon the difference in the amount of complex formed in parallel experiments, one experiment conducted in the presence of test compound and another experiment conducted in the absence of test compound. Alternatively, the amount of complex formed in the presence of the test compound can be compared against a historical value which is considered to be representative of the amount of complex formed under similar conditions except for the absence of test compound. A difference is typically considered to be “statistically significant” if the probability of the observed difference occurring by chance (the p-value) is less than some predetermined level. Thus, in a general sense, the phrase “statistically significant difference” refers to a difference that is greater than that which could simply be ascribed to experimental error. In a more formal sense, the phrase refers to a p-value that is <0.05, preferably <0.01 and most preferably <0.001.

In one specific example of a suitable screening method, screening can be carried out by contacting members from a library with one of the immune cell (e.g., a T cell or B cell) PDZ-domain polypeptides disclosed herein that is immobilized on a solid support and then collecting those library members that bind to the immobilized polypeptide. Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited hereinabove. Alternatively, the library members can be contacted with a domain from a cognate ligand protein (e.g., the C-terminus of a PL protein) that is immobilized to a support and collecting those members that bind to the immobilized polypeptide.

In other screening methods, the two-hybrid system for selecting interacting proteins in yeast (Fields and Song, 1989, Nature 340:245-246; Chien et al., 1991, Proc. Natl. Acad. Sci. USA 88:9578-9582) are used to identify molecules that specifically bind to a PDZ or PL domain-containing protein.

A large number of other screening methods are known and can be utilized in the screening methods provided herein. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

The foregoing screening methods can be utilized to screen essentially any type of natural, random or combinatorial library. By way of example, diversity libraries, such as random or combinatorial peptide or non-peptide libraries can be screened for molecules that specifically bind to PDZ domains in immune cells. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994.

In vitro translation-based libraries include, but are not limited to, those described in PCT Publication No. WO 91/05058 dated Apr. 18, 1991; and Mattheakis et al., 1994, Proc. Natl. Acad. Sci. USA 91:9022-9026.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Once a compound has been identified according to one of the foregoing screening methods, analogs based upon the identified compound can then be prepared. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to function as an agonist or antagonist of a particular interaction between a PDZ protein and its cognate ligand protein. Such compounds can then be subjected to further analysis to identify those compounds that appear to have the greatest potential as pharmaceutical compounds. Alternatively, analogs shown to have activity through the screening methods can serve as lead compounds in the preparation of still further analogs, which can be further screened by the methods disclosed herein. The cycle of screening, synthesizing analogs and rescreening can be repeated multiple times to further optimize the activity of the analog.

Further guidance on the synthesis of analog compounds and lead optimization is provided by, for example: Iwata, Y., et al. (2001) J. Med. Chem. 44:1718-1728; Prokai, L., et al. (2001) J. Med. Chem. 44:1623-1626; Roussel, P. et al., (1999) Tetrahedron 55:6219-6230; Bunin, B. A., et al. (1999) Ann. Rep. Med. Chem. 34:267-286; Venkatesh, S., et al. (2000) J. Pharm. Sci. 89:145-154; and Bajpai, M. and Adkinson, K. K. (2000) Curr. Opin. Drug Discovery and Dev. 3:63-71.

The following examples are provided to illustrate certain aspects of the methods and compositions that are described herein and are not to be construed to limit the scope of such methods and compositions.

### Example 1

### Inhibition of T Cell Activation by Mutation of PAG PDZ-Binding Motif

To test the role of the PDZ-binding motif present in PAG (ITRL) in T cell activation, we made two C-terminal mutants. In the mutant termed PAG C-ARA, we changed threonine and leucine to alanine; in PAG ΔPL the 3 most C-terminal residues were deleted, removing the PDZ ligand motif from PAG (FIG. 1). Plasmids encoding PAG, PAG C-ARA, and PAG ΔPL fusion proteins were transiently transfected into the Jurkat T cell leukemic line to assess their function, since T cell receptor signaling is dependent on the activity of the src kinases lck and fyn. In order to analyze TCR function, a Jurkat clone that contains a β-galactosidase reporter gene under the control of a triplicated form of the NFAT (nuclear factor of activated T cells) binding site was utilized. The activity of the NFAT transcription factor is a good indicator of T cell activation since its activity depends on activation of both critical arms of the T-Cell Receptor (TCR) signaling cascade: calcium mobilization and activation of the ras pathway (27). As a control in the experiment we utilized a member of the tumor necrosis factor family of receptors, DR6, whose cytoplasmic domain has been removed to prevent it from influencing TCR activity in any way. Twenty-four hours after transfection, cells were stimulated with anti-TCR antibodies (FIG. 2A) or Ionomycin+PMA (FIG. 2B) for 6 hours, then analyzed for β-galactosidase activity and expression of the N-terminal FLAG epitope by flow cytometry. Results are expressed as the percentage of activated cells within the three designated populations: (a) Flag (−) or untransfected cells, and those that (b) expressed either low-intermediate, or (c) high levels of the transfected proteins, Flag (+).

As expected, expression of the truncated DR6 protein in Jurkat cells has no effect on TCR-mediated activation of NFAT (FIGS. 2A and 2B). In contrast, cells expressing the transfected wild type PAG showed a 30% reduction in NFAT activity, while cells that failed to express the protein were unaffected. Both mutations in the PDZ binding motif resulted in enhanced inhibition to 40%, indicating that the PDZ interaction is important for optimal TCR activation. Therefore, blocking the binding of PAG and its PDZ-binding partners would be expected to suppress T cell responses (see FIG. 3).

### Example 2

### Cloning of Human Shank 3 PDZ Domain

Human shank 3 was cloned in the following manner. An expressed sequence tag (EST) was identified by a BLAST search of the human ESTs in Genebank using rat Shank 3 sequence (gi:11067398). Oligonucleotides based on the EST sequence (736 SHF—TGGATCCTTGAGGAGAAGACGGTG; 737 shr—TGCAATTGTCGTCGGGGTCCAGATTC) were designed and the PDZ of human Shank was amplified by standard methods using PCR from Jurkat E6 T cell line cDNA. Amplified fragments were digested with BamHI and MfeI and cloned into the BamHI and EcoRI sites of pGEX-3X for expression (Amersham-Pharmacia).

### Example 3

### Expression of Human Shank3 PDZ Domain in Bacterial Cells

The PCR fragment corresponding to the PDZ domain of human Shank3 was cloned in frame into the pGEX-3X vector (Amersham-Pharmacia) to generate a GST-Shank3 fusion vector. The GST fusion protein was expressed by IPTG induction in DH5α bacterial cells and purified using glutathione sepharose chromatography according to manufacture's instructions (Pharmacia). Purified protein was analyzed by SDS-PAGE and dialyzed against storage buffer (PBS with 25% glycerol) and stored at −20° C. (short term) or −80° C. (long term).

### Example 4

### Identification of Ligand Interactions with the PDZ Domains of Shank 1 and Shank 3

The binding of various ligands to Shank 1 and Shank 3 PDZ domain was assessed using a modified ELISA. The binding of GST fusion proteins that contained the PDZ domain of human Shank 1 and Shank 3 to biotinylated peptides corresponding to the C-terminal 20 amino acids of diverse proteins was detected through a colorimetric assay using avidin-HRP to bind the biotin and a peroxidase substrate (G-assay, below; see also PCT Publications WO 00/69896, WO 00/69898 and WO 00/69897). By titrating the amount of peptide and protein added to these reactions, dissociation constants (Kd) were determined as an indication of relative affinity (see also, PCT Publications WO 00/69896, WO 00/69898 and WO 00/69897).

A. Peptide Purification

Peptides representing the C-terminal 8 or 20 amino acids of proteins were synthesized by standard FMOC chemistry. The peptides were biotinylated on request. Peptides were purified by reverse phase high performance liquid chromatography (HPLC) using a Vydac 218TP C18 Reversed Phase column having the dimensions of 10×25 mm, 5 um. Approximately 40 mg of the peptide were dissolved in 2.0 ml of 50:50 ratio of acetonitrile/water+0.1% tri-fluoro acetic acid (TFA). This solution was then injected into the HPLC machine through a 25 micron syringe filter (Millipore). Buffers used to obtain separation were (A) Distilled water with 0.1% TFA and (B) 0.1% TFA with acetonitrile. Gradient segment setup is listed in the Table I below.

TABLE I

| | | | low rate |
|---|---|---|---|
| ime | | | (ml/min) |
| | 6% | % | .00 |
| 0 | 00% | 00% | .00 |
| 5 | 00% | 00% | .00 |
| 0 | 6% | % | .00 |

The separation occurs based on the nature of the peptides. A peptide of hydrophobic nature will elute off later than a peptide having a hydrophilic nature. Based on these principles, the peak containing the “pure” peptide is collected. Their purity is checked by Mass Spectrometer (MS). Purified peptides are lyophilized for stability and later use.

B. “G” Assay for Identification of Interactions Between Peptides and Fusion Protein

1. Reagents and Materials

Nunc Polysorp 96 well Immuno-plate (Nunc cat#62409-005). (Maxisorp plates have been shown to have higher background signal)

PBS pH 7.4 (Gibco BRL cat#16777-148) or AVC phosphate buffered saline, 8 g NaCl, 0.29 g KCl, 1.44 g Na₂HPO4, 0.24 g KH₂PO₄, add H₂O to 1 L and pH 7.4; 0.2μ filter

2% BSA/PBS (10 g of bovine serum albumin, fraction V (ICN Biomedicals cat #IC15142983) into 500 ml PBS

Goat anti-GST mAb stock @ 5 mg/ml, store at 4° C., (Amersham Pharmacia cat #27-4577-01), dilute 1:1000 in PBS, final concentration 5 μg/ml

HRP-Streptavidin, 2.5 mg/2 ml stock stored at 4° C. (Zymed cat #43-4323), dilute 1:2000 into 2% BSA, final concentration at 0.5 μg/ml

Wash Buffer, 0.2% Tween 20 in 50 mM Tris pH 8.0

TMB ready to use (Dako cat #S1600)

1M H₂SO₄

12w multichannel pipettor,

50 ml reagent reservoirs,

15 ml polypropylene conical tubes

C. Protocol

1) Coat plate with 100 ul of 5 ug/ml goat anti GST, 0/N @ 4° C.
2) Dump coating antibodies out and tap dry
3) Blocking—Add 200 ul per well 2% BSA, 2 hrs at 4° C.
4) Prepare proteins in 2% BSA

(2 ml per row or per two columns)

5) 3 washes with cold PBS (must be cold through entire experiment)

(at last wash leave PBS in wells until immediately adding next step)

6) Add proteins at 50 ul per well on ice (1 to 2 hrs at 4° C.)
7) Prepare peptides in 2% BSA (2 ml/row or/columns)
8) 3× wash with cold PBS
9) Add peptides at 50 ul per well on ice (time on/time off)
keep on ice after last peptide has been added for 10 minutes exactly
place at room temp for 20 minutes exactly
   10) Prepare 12 ml/plate of HRP-Streptavidin (1:2000 dilution in 2% BSA)
   11) 3× wash with cold PBS
   12) Add HRP-Streptavidin at 100 ul per well on ice, 20 minutes at 4° C.
   13) Turn on plate reader and prepare files
   14) 5× washes, avoid bubbles
   15) Using gloves, add TMB substrate at 100 ul per well
incubate in dark at room temp
check plate periodically (5, 10, and 20 minutes)
take early readings, if necessary, at 650 nm (blue)
at 20 minutes, stop reaction with 100 ul of 1M H2SO4
take last reading at 450 nm (yellow)

A450 readings representing interactions between PDZ domains and their ligands are given a classification of 0 to 5. Classifications: 0—interaction is less than 10 uM; 1—A450 between 0 and 1; 2—A450 between 1 and 2; 3—A450 between 2 and 3; 4—A450 between 3 and 4; 5—A450 of 4 or more observed 2 or more times.

D. Results

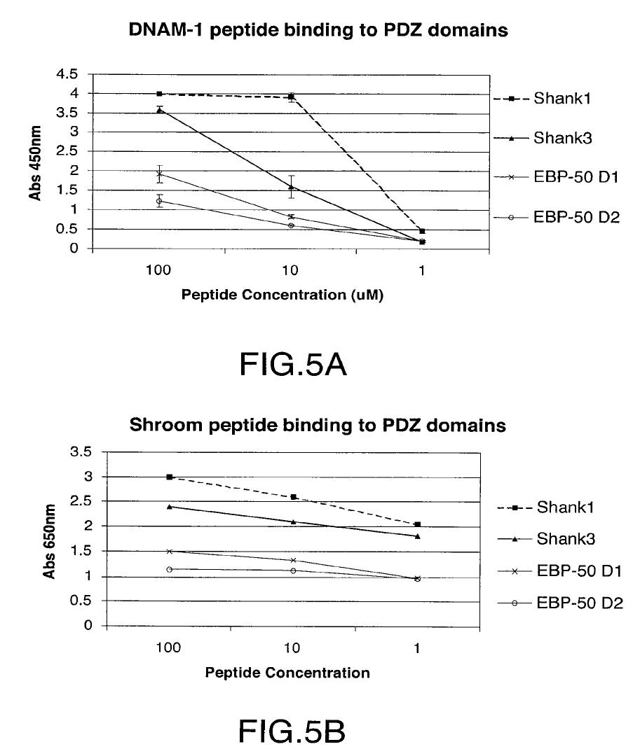

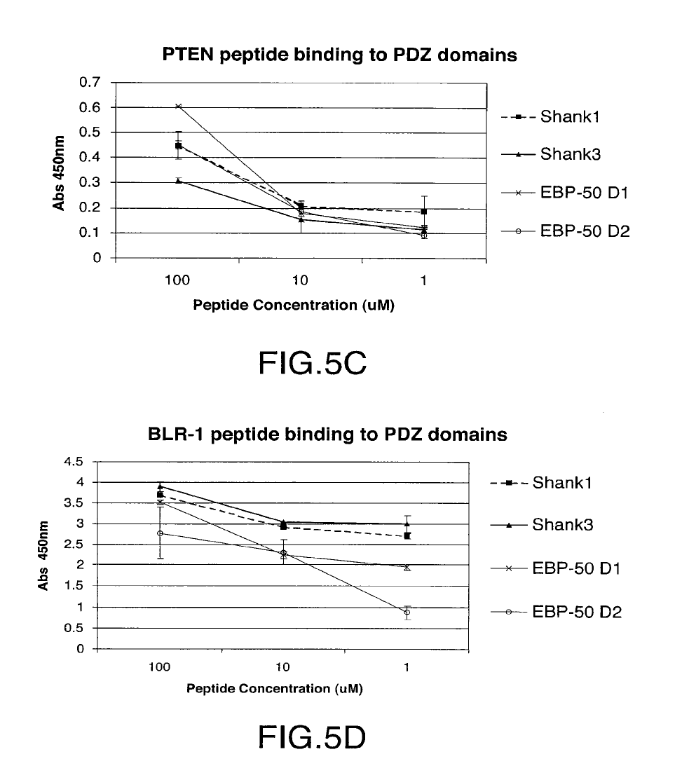

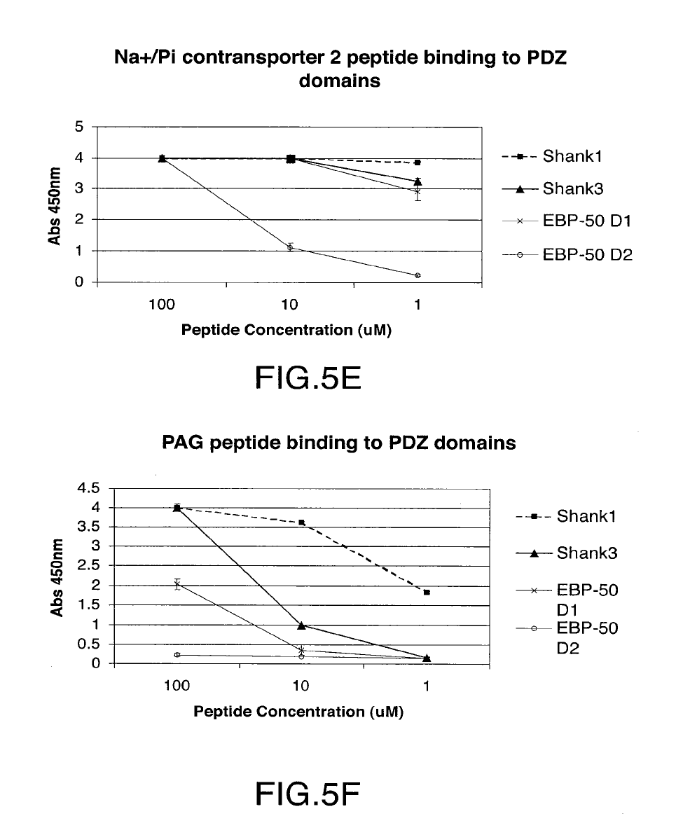

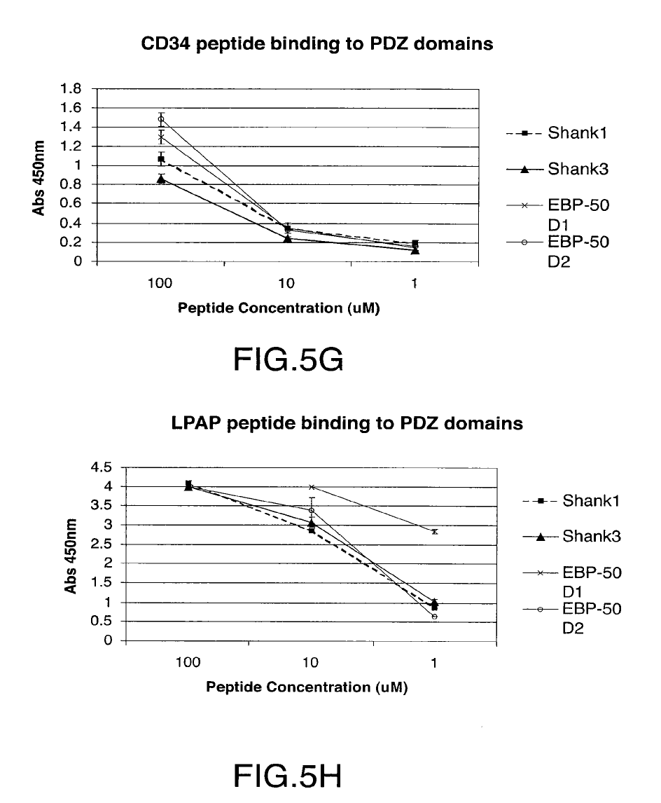

The C-terminal peptides of LPAP and PAG were tested against 156 PDZ domains. Results are shown in Table II below and FIGS. 5A-51. Shank1, Shank3 and KIAA807 were observed to have the highest affinity interactions with the PL domain of PAG. Shank1 PDZ domain potential interactions were also tested against 114 C-terminal peptides corresponding to PLs of various biological proteins (Table III and FIGS. 5A-51). Binding partners identified include DNAM-1 (category 2), HPVE6 33 (modified; category 2), CD128B (category 3), LPAP (category 2), Neuroligin (category 2), PTEN (category 3), Na⁺/Pi co-transporter (category 4), PAG (category 5), and KIAA1481 (category 5). Interaction of human Shank3 PDZ domain was tested with all peptides that bound Shank1. The results displayed very similar binding patterns, including the high-affinity binding to PAG (category 5).

The C-terminal peptide of PAG was also tested against PDZ domains 1 and 2 of EBP50. Results show that the interaction of PAG with PDZ domain 1 of EBP50 is a category 5 interaction. The PAG interactions with Shank 1, Shank 3, KIA1481 and EBP50 PDZ domain 1 were titrated in parallel (FIGS. 5A-51).

TABLE II

| PL | PDZ | PDZ Domain | Classification |
|---|---|---|---|
| LPAP | KIAA0807(S) | 1 | 5 |
| LPAP | KIAA1526 | 1 | 1 |
| LPAP | Atrophin-1 Inter. Prot. | 5 | 2 |
| LPAP | BAI-1 | 2 | 2 |
| LPAP | KIAA807 | | 5 |
| LPAP | Mint 1 | 2 | 1 |
| LPAP | Mint 1 | 1, 2 | 1 |
| LPAP | FLJ 00011 | 1 | 4 |
| LPAP | FLJ 10324 | 1 | 1 |
| LPAP | GRIP1 | 3 | 1 |
| LPAP | PDZK1 | 2, 3, 4 | 3 |
| LPAP | NOS1 | 1 | 1 |
| LPAP | hAPXL | 1 | 1 |
| LPAP | HEMBA 1003117 | 1 | 1 |
| LPAP | PIST | 1 | 1 |
| LPAP | PTPL-1 | 2 | 1 |
| LPAP | KIAA0147 | 1 | 3 |
| LPAP | SHANK | 1 | 2 |
| LPAP | KIAA0316 | 1 | 1 |
| LPAP | KIAA0382 | 1 | 5 |
| LPAP | TIP1 | 1 | 5 |
| LPAP | Unnamed Protein | 2 | 3 |
| PAG | KIAA0807(S) | 1 | 5 |
| PAG | Atrophin-1 Inter. Prot. | 5 | 1 |
| PAG | KIAA807 | | 5 |
| PAG | FLJ 00011 | 1 | 3 |
| PAG | PDZK1 | 2, 3, 4 | 1 |
| PAG | Outer Membrane | 1 | 2 |
| PAG | hAPXL | 1 | 2 |
| PAG | PIST | 1 | 1 |
| PAG | SHANK | 1 | 5 |
| PAG | KIAA0316 | 1 | 1 |
| PAG | KIAA0382 | 1 | 1 |

Table II shows a partial list of PDZ domains that interact with the C-terminus (PDZ ligand or PL) of LPAP and PAG. The first column displays the PL gene name and the second displays the PDZ domain-containing protein used to assess binding. The third column lists the specific PDZ domain that showed a measurable interaction in this assay (number from the amino terminus of the protein; see also PCT Publications WO 00/69898, WO 00/69897 and WO 0069896). The fourth column, ‘classification’, refers to the strength of binding. Classifications: 1—A450 between 0 and 1; 2—A450 between 1 and 2; 3—A450 between 2 and 3; 4—A450 between 3 and 4; 5—A450 of 4 or more observed 2 or more times.

TABLE III

| PDZ | Domain | PL | Classification |
|---|---|---|---|
| DLG1 | 1, 2 | a-actinin 2 | 1 |
| DLG1 | 1, 2 | Adenovirus E4 Type9 | 5 |
| DLG1 | 1, 2 | APC- adenomatous polyposis | 5 |
| | | coli protein | |
| DLG1 | 1, 2 | catenin - delta 2 | 3 |
| DLG1 | 1, 2 | CD95 (fas) | 2 |
| DLG1 | 1, 2 | claudin 10 | 1 |
| DLG1 | 1, 2 | DNAM-1 | 1 |
| DLG1 | 1, 2 | ErbB-4 receptor | 1 |
| DLG1 | 1, 2 | GluR5-2 (rat) | 5 |
| DLG1 | 1, 2 | HPV E6 #35 (modified) | 5 |
| DLG1 | 1, 2 | HPV E6 #66 (modified) | 5 |
| DLG1 | 1, 2 | Kir2.1 (inwardly rect. K+ | 2 |
| | | channel) | |
| DLG1 | 1, 2 | Nedasin (s-form) | 3 |
| DLG1 | 1, 2 | Neuroligin | 2 |
| DLG1 | 1, 2 | NMDA Glutamate Receptor | 5 |
| | | 2C | |
| DLG1 | 1, 2 | NMDA R2C | 1 |
| DLG1 | 1, 2 | PDZ-binding kinase (PBK) | 1 |
| DLG1 | 1, 2 | RGS12 (regulator of G-protein | 1 |
| | | signaling 12 | |
| DLG1 | 1, 2 | SSR4_HUMAN | 1 |
| DLG1 | 1, 2 | Tax | 5 |
| DLG1 | 1 | Adenovirus E4 Type9 | 4 |
| DLG1 | 1 | catenin - delta 2 | 1 |
| DLG1 | 1 | GluR5-2 (rat) | 2 |
| DLG1 | 1 | HPV E6 #35 (modified) | 5 |
| DLG1 | 1 | HPV E6 #66 (modified) | 4 |
| DLG1 | 1 | NMDA Glutamate Receptor | 5 |
| | | 2C | |
| DLG1 | 1 | Tax | 5 |
| DLG1 | 2 | a-actinin 2 | 1 |
| DLG1 | 2 | Adenovirus E4 Type9 | 5 |
| DLG1 | 2 | catenin - delta 2 | 2 |
| DLG1 | 2 | CD95 (fas) | 1 |
| DLG1 | 2 | CITRON protein | 2 |
| DLG1 | 2 | GluR5-2 (rat) | 2 |
| DLG1 | 2 | GLUR7 (metabotropic | 1 |
| | | glutamate receptor) | |
| DLG1 | 2 | HPV E6 #35 (modified) | 5 |
| DLG1 | 2 | HPV E6 #66 (modified) | 5 |
| DLG1 | 2 | Kir2.1 (inwardly rect. K+ | 1 |
| | | channel) | |
| DLG1 | 2 | NMDA Glutamate Receptor | 5 |
| | | 2C | |
| DLG1 | 2 | Tax | 5 |
| DLG1 | 3 | ephrin B2 | 1 |
| DLG1 | 3 | GluR5-2 (rat) | 1 |
| DLG1 | 3 | HPV E6 #35 (modified) | 3 |
| DLG1 | 1, 2 | GLUR2 (glutamate receptor 2 | 2 |
| DLG1 | 2 | GLUR2 (glutamate receptor 2 | 1 |
| DLG1 | 1 | Clasp-2 | 1 |
| DLG1 | 2 | Clasp-2 | 1 |
| DLG1 | 1 | HPV E6 33 (modified) | 3 |
| DLG1 | 2 | HPV E6 33 (modified) | 5 |
| DLG1 | 1 | HPV E6 58 (modified) | 5 |
| DLG1 | 2 | HPV E6 58 (modified) | 5 |
| DLG2 | 1 | GluR5-2 (rat) | 1 |
| DLG2 | 1 | HPV E6 #35 (modified) | 5 |
| DLG2 | 1 | HPV E6 #66 (modified) | 1 |
| DLG2 | 1 | NMDA Glutamate Receptor | 4 |
| | | 2C | |
| DLG2 | 1 | Tax | 2 |
| DLG2 | 2 | Adenovirus E4 Type9 | 5 |
| DLG2 | 2 | catenin - delta 2 | 1 |
| DLG2 | 2 | CD95 (fas) | 1 |
| DLG2 | 2 | GluR5-2 (rat) | 1 |
| DLG2 | 2 | HPV E6 #35 (modified) | 5 |
| DLG2 | 2 | HPV E6 #66 (modified) | 5 |
| DLG2 | 2 | Kir2.1 (inwardly rect. K+ | 1 |
| | | channel) | |
| DLG2 | 2 | NMDA Glutamate Receptor | 5 |
| | | 2C | |
| DLG2 | 2 | Tax | 5 |
| DLG2 | 2 | GLUR2 (glutamate receptor 2 | 1 |
| DLG2 | 1 | HPV E6 33 (modified) | 1 |
| DLG2 | 2 | HPV E6 33 (modified) | 3 |
| DLG2 | 2 | HPV E6 58 (modified) | 5 |
| DLG5 | 2 | ephrin B2 | 1 |
| DLG5 | 2 | A2AA_HUMAN (modified) | 1 |
| NeDLG | 1 | Adenovirus E4 Type9 | 1 |
| NeDLG | 1 | HPV E6 #35 (modified) | 5 |
| NeDLG | 1 | HPV E6 #66 (modified) | 1 |
| NeDLG | 1 | NMDA Glutamate Receptor | 2 |
| | | 2C | |
| NeDLG | 2 | Adenovirus E4 Type9 | 5 |
| NeDLG | 2 | ephrin B2 | 2 |
| NeDLG | 2 | GluR5-2 (rat) | 1 |
| NeDLG | 2 | HPV E6 #35 (modified) | 5 |
| NeDLG | 2 | HPV E6 #66 (modified) | 4 |
| NeDLG | 2 | NMDA Glutamate Receptor | 5 |
| | | 2C | |
| NeDLG | 2 | Tax | 5 |
| NeDLG | 3 | catenin - delta 2 | 1 |
| NeDLG | 3 | CITRON protein | 3 |
| NeDLG | 3 | ephrin B2 | 1 |
| NeDLG | 3 | GluR5-2 (rat) | 2 |
| NeDLG | 3 | HPV E6 #35 (modified) | 5 |
| NeDLG | 3 | Neuroligin | 1 |
| NeDLG | 3 | NMDA Glutamate Receptor | 1 |
| | | 2C | |
| NeDLG | 3 | Tax | 5 |
| NeDLG | 1, 2 | Tax | 5 |
| NeDLG | 1, 2 | PDZ-binding kinase (PBK) | 1 |
| NeDLG | 1, 2 | NMDA R2C | 2 |
| NeDLG | 1, 2 | NMDA Glutamate Receptor | 5 |
| | | 2C | |
| NeDLG | 1, 2 | Neuroligin | 1 |
| NeDLG | 1, 2 | Nedasin (s-form) | 2 |
| NeDLG | 1, 2 | Kir2.1 (inwardly rect. K+ | 1 |
| | | channel) | |
| NeDLG | 1, 2 | HPV E6 #66 (modified) | 5 |
| NeDLG | 1, 2 | HPV E6 #35 (modified) | 5 |
| NeDLG | 1, 2 | GluR5-2 (rat) | 5 |
| NeDLG | 1, 2 | ErbB-4 receptor | 1 |
| NeDLG | 1, 2 | DNAM-1 | 2 |
| NeDLG | 1, 2 | CD95 (fas) | 1 |
| NeDLG | 1, 2 | APC- adenomatous polyposis | 4 |
| | | coli protein | |
| NeDLG | 1, 2 | Adenovirus E4 Type9 | 5 |
| NeDLG | 1, 2 | GLUR2 (glutamate receptor 2 | 2 |
| NeDLG | 1, 2 | Clasp-2 | 2 |
| NeDLG | 2 | Clasp-2 | 1 |
| NeDLG | 1, 2 | HPV E6 33 (modified) | 5 |
| NeDLG | 1 | HPV E6 33 (modified) | 1 |
| NeDLG | 2 | HPV E6 33 (modified) | 2 |
| NeDLG | 3 | HPV E6 33 (modified) | 1 |
| NeDLG | 1, 2 | HPV E6 58 (modified) | 5 |
| NeDLG | 1 | HPV E6 58 (modified) | 1 |
| NeDLG | 2 | HPV E6 58 (modified) | 5 |
| NeDLG | 3 | HPV E6 58 (modified) | 2 |
| rat SHANK | 1 | a-actinin 2 | 1 |
| 3 | | | |
| rat SHANK | 1 | Na+/Pi cotransporter 2 | 4 |
| 3 | | | |
| SHANK | 1 | CDw128B | 3 |
| SHANK | 1 | LPAP | 2 |
| SHANK | 1 | PAG | 5 |
| SHANK | 1 | a-actinin 2 | 1 |
| SHANK | 1 | BLR-1 | 1 |
| SHANK | 1 | CD34 | 1 |
| SHANK | 1 | CFTCR (cystic fibrosis | 1 |
| | | transmembrane conductance | |
| | | regulator) | |
| SHANK | 1 | CD68 | 1 |
| SHANK | 1 | DNAM-1 | 2 |
| SHANK | 1 | Dock2 | 1 |
| SHANK | 1 | KIA 1481 | 5 |
| SHANK | 1 | Na+/Pi cotransporter 2 | 4 |
| SHANK | 1 | Neuroligin | 2 |
| SHANK | 1 | PTEN | 3 |
| SHANK | 1 | zona occludens 3 (ZO-3) | 1 |
| SHANK | 1 | SSTR2 (somatostatin recepor 2) | 1 |
| SHANK | 1 | GABA transporter 3 | 1 |
| SHANK | 1 | Clasp-5 | 1 |
| SHANK | 1 | HPV E6 33 (modified) | 2 |

Table III shows a partial list of PDZ ligands that interact with the PDZ domains of DLG1, DLG2, DLG5, NeDLG, and SHANK. The first column displays the PDZ gene name and the second displays the domain or domains contained in the fusion used to assess binding. The third column names the PDZ ligand that showed a measurable interaction in this assay. The fourth column, ‘classification’, refers to the strength of binding. Classifications: 1—A450 between 0 and 1; 2—A450 between 1 and 2; 3—A450 between 2 and 3; 4—A450 between 3 and 4; 5—A450 of 4 or more observed 2 or more times.

### Example 5

### Presence of PDZ Domain Containing Proteins in Human T Cells

Expression of several proteins containing PDZ domains was analyzed on Jurkat T cells by Western blot. The Jurkat subclone used in this work is an isolate that has been engineered to express SV40 large T antigen and several inducible cell surface proteins and selected for high (>90%) expression of CD3 (N. Jacobson, unpublished). Jurkat cell lysates were probed with antibodies that recognize hDlg1, Dvl1, Dvl2, PICK1, hScribble1 (Santa Cruz Biotechnology, Inc., Santa Cruz, Calif.), PSD95, GRIP (Upstate Biotechnology Inc., Lake Placid, N.Y.), CASK, (Zymed, So. San Francisco, Calif.); Chapsyn, (Calbiochem), Shank (provided by Dr Morgan Sheng) and CNK (Transduction Laboratories, Lexington, Ky.). Results show that CASK, Dlg, Dvl2, Pick1, CNK, Shank, GRIP and PSD-95 were expressed on human T cells and others like Chapsyn and Dvl1 were not expressed in this specific cell line (FIG. 6A).

### Example 6

### Presence of PDZ-Containing Proteins on T Cells Lipid Rafts

Cytoplasmic (C), membrane (M) and detergent insoluble (D) fractions were prepared by isopycnic sucrose gradient centrifugation, from Jurkat T cells stimulated or not with anti-CD3 antibody, OKT3. The presence of PDZ containing proteins and signaling molecules involved in T cell activation in the different fractions was analyzed by Western blot (FIG. 6B). Actin binding proteins WASP and IQGAP are predominantly represented in the cytosolic and membrane fractions, whereas the concentrations of PKC-θ and GADS increase in the detergent insensitive glycolipid-enriched compartment (DIG) after activation. LFA-1 is enriched in membrane and DIG fractions independent of TCR activation. The PDZ proteins hDlg1 and CASK are concentrated in lipid rafts, whereas PDZ proteins GRIP and Dvl2 are excluded from the detergent insoluble fraction.

### Example 7

### Dlg Association with Lipid Rafts does not Require Tyrosine Kinase p56 Lck

The presence of Dlg in lipid rafts was analyzed by Western blot in Jurkat T cells and in a Jurkat mutant that lacks p56 Lck. As shown in FIG. 7B, hDlg1 is associated with the detergent insoluble membrane fraction or lipid rafts in both Lck negative Jurkat cells and parental Jurkat cells. Therefore, Dlg association with lipid rafts is not dependent on the tyrosine kinase Lck.

### Example 8

### Dlg Association with Tyrosine Phosphorylated Proteins after TCR Stimulation

To identify the proteins that associate with Dlg upon TCR stimulation, lysates of Jurkat T cells activated with anti-CD3 plus anti-CD28 or with H2O2 (activates Lck but not TCR) were prepared. Dlg and proteins interacting with Dlg were immunoprecipitated using antibodies against Dlg. Dlg-immunoprecipitates were analyzed for phosphotyrosine-containing proteins by Western blotting with mAb 4G10. In addition, Western blots were probed with antibodies against molecules known to be phosphorylated upon T cell activation. Results, shown in FIGS. 7D and 9-10, identified the phosphoproteins associated to Dlg as Lck, CD3ζ, LAT, Cbl, CAMKII, LFA-1, and CASK.

### Example 9

### Structural Requirements in Dlg for Association with Lck, CD3ζ, LAT, and Cbl

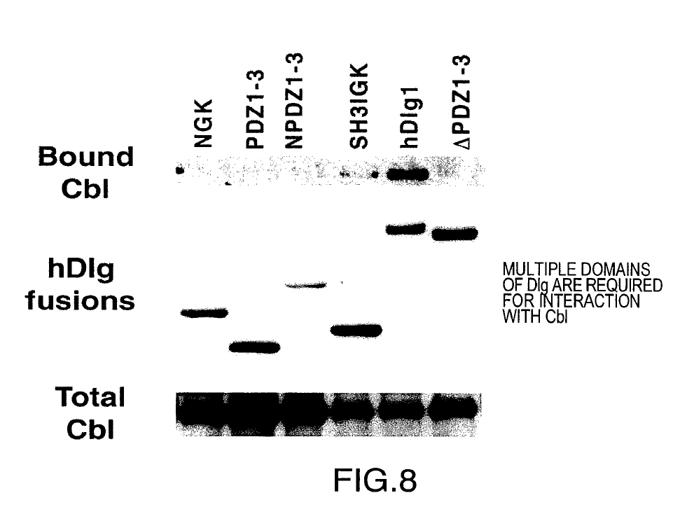

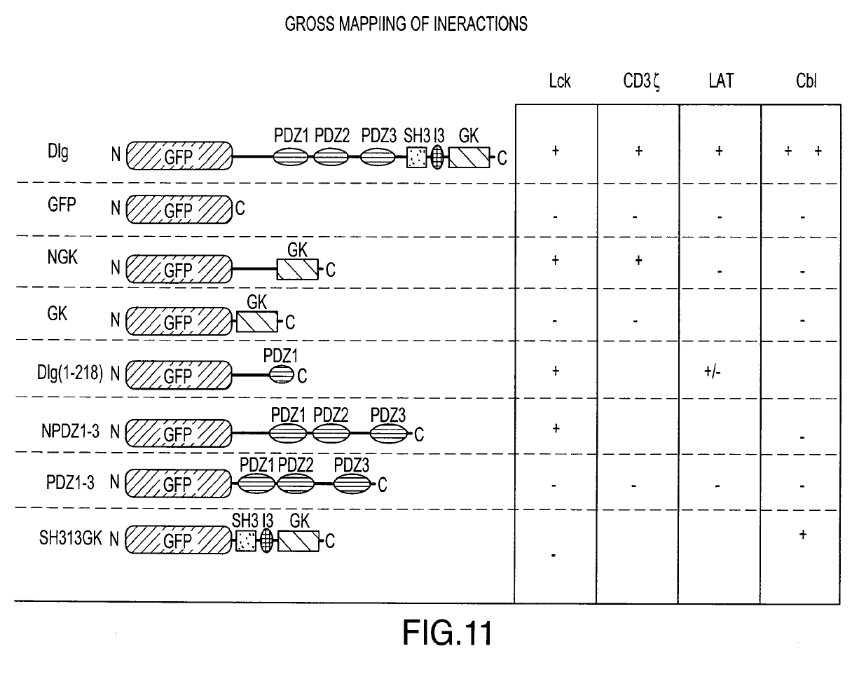

Several truncation mutants of Dlg were introduced into a green fluorescent protein (GFP)-vector and transfected into Jurkat cells (see FIG. 11 for a schematic representation of which Dlg domains are included for the various mutants). The GFP fusion proteins were then analyzed for their ability to bind Lck, CD3ζ, LAT, and Cbl by anti-EGFP immunoprecipitation and Western blotting. Results demonstrate that multiple domains of Dlg are required for interaction with Cbl (FIG. 8). The minimal requirements for Dlg association to bind Lck, CD3ζ, LAT, are summarized in FIG. 11.

### Example 10

### Association of Dlg with the Actin Cytoskeleton

Total, membrane (Memb) and cytosolic (Cyt) fractions were prepared from Jurkat T cells, either unstimulated or stimulated with OKT3 mAb. hDlg, CASK and associated proteins were immunoprecipitated from these cellular fractions using antibodies against hDlg and CASK (see Example 5). Western blots were then performed on these fractions with an actin-specific antibody (ICN). Results show that T cell activation promotes the association of membrane-associated Dlg with the actin cytoskeleton (FIG. 7C).

The GFP/hDlg fusion protein (Wu et al, 1998) was then transfected into Jurkat and 293T cells to examine colocalization of Dlg and actin. Cells were stained with anti-actin antibodies (red) and analyzed by immunofluorescence microscopy. Results showed cortical colocalization of actin and Dlg 1-GFP in 293T cells and Jurkat cells activated with anti-CD3.

### Example 11

### Dlg1 Induces Apoptosis in Jurkat T Cells

Jurkat cells were electroporated with vectors encoding Dlg1-GFP, the internal deletion mutant, Dlg1NGK-GFP (consisting of residues 1-186, the N-terminus, fused to 683-906, the guanylate kinase domain), CASK-GFP or GFP alone and the GFP intensity was measured by flow cytometry (FIGS. 12-13) in the presence and absence of zVAD, an inhibitor of apoptosis. Overexpression of Dlg1 itself, and Dlg1NGK resulted in a significant induction of cell death, evidenced by the decrease in percentage of GFP positive cells in the total surviving pool. Constructs encoding Dlg1-GFP, Dlg1NGK-GFP, and GFP produced similar levels of fluorescence in 293 T cells, indicating that the toxicity induced by the former constructs is cell-specific. Therefore, overexpression of merely the N-terminus and guanylate kinase domains of Dlg is enough to result in cell death. Inclusion of the 3 PDZ domains of Dlg still resulted in an increase in cell death, although to a lesser extent than the NGK construct that lacks the PDZ domains.

### Example 12

### hDlg Attenuation of TCR-Mediated Mobilization of Calcium

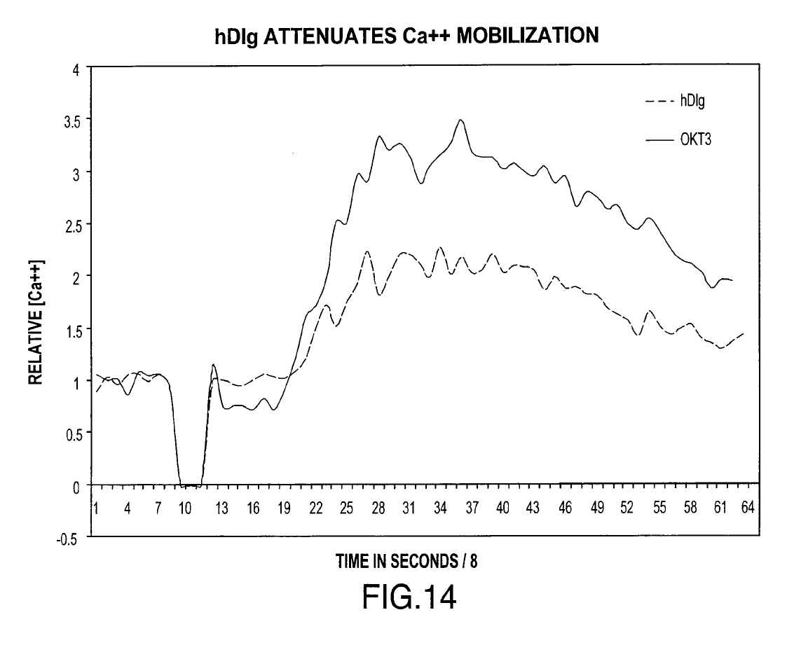

Jurkat T cells untransfected or transfected with hDlg were loaded with a calcium-sensitive fluorescent dye and stimulated with OKT3 antibody. Calcium mobilization of was analyzed by flow cytometry. Jurkat T cells expressing hDlg show reduced calcium mobilization after TCR activation (FIG. 14), indicating that overexpression of Dlg reduces the ability of cells to become activated after stimulation.

### Example 13

### Analysis of CASK and Actin Colocalization

CASK is a PDZ domain-containing protein that is expressed in lymphocytes. The domain structure of CASK is shown in FIG. 15A along with proteins that are known to interact with those domains.

Colocalization of CASK and actin was analyzed in 293T cells. A green fluorescent protein-CASK fusion (GFP-CASK) was introduced into 293T cells by standard calcium phosphate precipitation methods. Cells were fixed, permeabilized and examined for green fluorescence indicative of GFP-CASK localization, and red fluorescent using a tagged antibody against actin (see Example 10). Unlike hDlg the majority of the transfected GFP-CASK does not colocalize with actin under these conditions.

### Example 14

### Cask Associated Proteins after TCR Stimulation of Jurkat T Cells

CASK interactions were examined in Jurkat T cells. Jurkat cells were unstimulated (−) or stimulated with OKT3 (+), lysed, and fractionated into cytoplasmic (C) and membrane (M) fractions by standard methods (detergent and centrifugation). CASK was immunoprecipitated from these fractions and its association with the indicated proteins analyzed by Western blot using antibodies specific to the proteins listed to the left or right of the lanes shown in FIG. 16A. The results show that CASK is localized to both cytoplasmic and membrane fractions regardless of activation by OKT3. The results further show that vav and CDC42 are associated with CASK, especially post-activation in the case of CDC42. However, we did not observe association of LFA-1, cbl or SLP-76 with CASK.

Interactions between CASK and other signaling molecules were analyzed by co-transfection and immunoprecipitation experiments in 293T cells (FIG. 16B). A CASK construct was made with an AU1 epitope at the C-terminus to use for immunoprecipitation (FIG. 15B). This construct was co-transfected into 293T cells with either zap70, cbl, hDlg1 or vav. Total lysates of the co-transfected cells were run along with an immunoprecipitate using the anti-Au1 antibody. Each blot was probed for the co-transfected protein (FIG. 16B). We observe that zap70, hDlg1 and vav can be co-immunoprecipitated with CASK, but that cbl did not co-immunoprecipitate with CASK.

### Example 15

### Activation-Dependent Association of Signaling Molecules with CASK

Jurkat cells were stimulated for 0, 3, 7, or 10 minutes with OTK3 mAb, lysed, and CASK immunoprecipitates analyzed for phosphotyrosine content with the mAb 4G10 (FIG. 17, upper panel) or for the presence of PKCθ or ZAP-70 by Western blot (FIG. 17, lower panel). As can be seen, PKCθ and ZAP 70 are minimally associated with CASK in resting cells but they associate following activation.

### Example 16

### Structural Requirements for CASK and Cdc42/rac Interaction

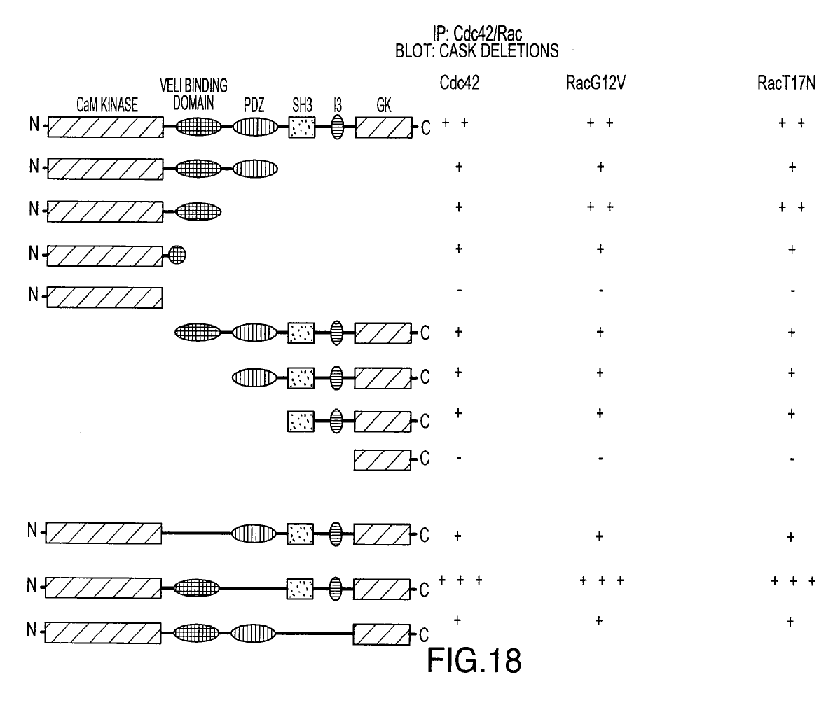

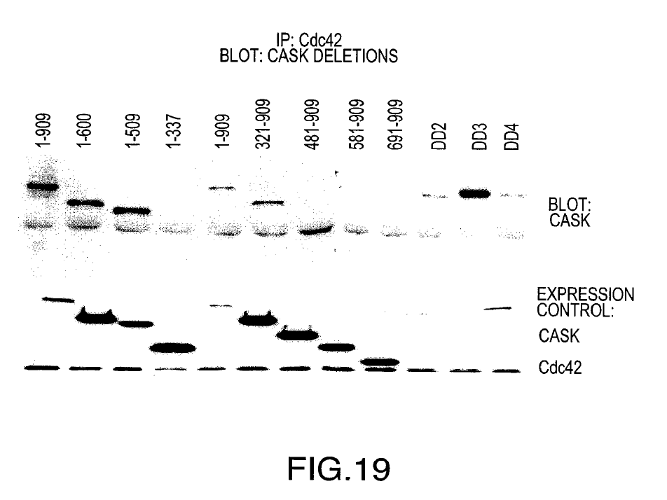

A schematic representation of the assay used to define the interaction requirements for CASK association with the Cdc42/rac GTPase is provided in FIG. 15B. An N-terminal FLAG-tagged version of Cdc42/rac was co-transfected with a series of C-terminal Au1-tagged CASK deletion mutants (FIG. 18). Cdc42/rac was precipitated via the FLAG epitope and association with partial CASK constructs was monitored by immunoblotting with an Au1-specific mAb. A summary of binding data of the different CASK mutants, is show in FIG. 18. A constitutively activated mutant of Cdc42/rac (RacG12V) or a dominant-negative variant (RacT17N) exhibited no altered pattern of associations with CASK (FIG. 18). FIG. 19 shows the results of Flag-Ccd42/rac association to CASK proteins (the numbers refer to the amino acids present in the CASK constructs) after immunoprecipitation with anti-Flag antibody, followed by Western blotting with anti-Au1.

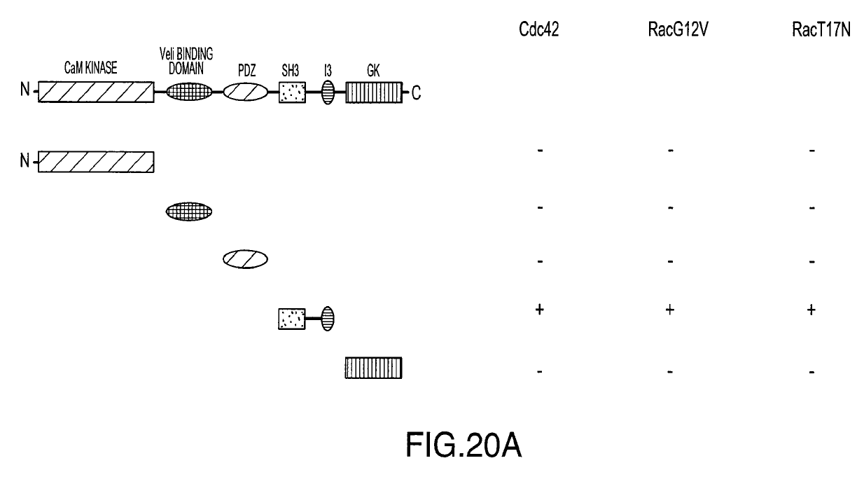

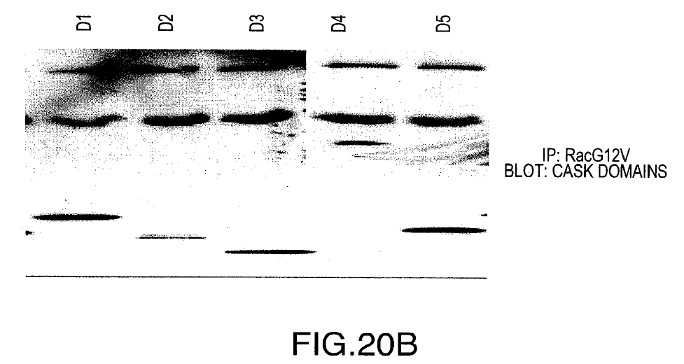

Constructs containing the isolated domains within CASK (FIG. 20A) were transfected into Jurkat T cells. Lysates were immunoprecipitated with anti-rac antibodies, and analyzed for CASK association by Western blotting (D1-5 in FIG. 20B, refer to domains depicted in FIG. 20A). Results, summarized in FIG. 20A (right panel), show Cdc42/rac association with the SH3-13 domain of CASK. Activated (RacG12V) or dominant-negative (RacT17N) forms of rac also associate with the SH3-13 domain of CASK. Thus, CASK binds various forms of activated Ras, while, in contrast, hDlg does not. This association appears to require residues between 337 and 600 of CASK.

### Example 17

### Opposite Effects of Dlg1 and CASK Expression on Transcriptional Activity in Jurkat Cells

Jurkat T cells were co-transfected with the reporter constructs NFAT-luciferase or SV40NFκB-luciferase, and plasmids expressing Vav1, GFP, and either Dlg1-GFP or CASK-GFP fusion constructs. Transfected cells were either left untreated or stimulated with anti-CD3 antibody. The cells were lysed and luciferase activity was measured.

Relative to control (GFP), CASK-GFP activates basal NF-κB activity. In contrast, Dlg1-GFP inhibits basal NF-κB activity (FIG. 21B). As for NF-κB, overexpression of CASK-GFP induces basal NFAT activity and enhances Vav1-induced NFAT activation; however, Dlg1-GFP inhibits Vav1-induced NFAT induction (FIG. 21A).

### Example 18

### Intracellular Ca+2 Mobilization in Jurkat T Cells Induced by Crosslinking of a CD16: 7: CASK

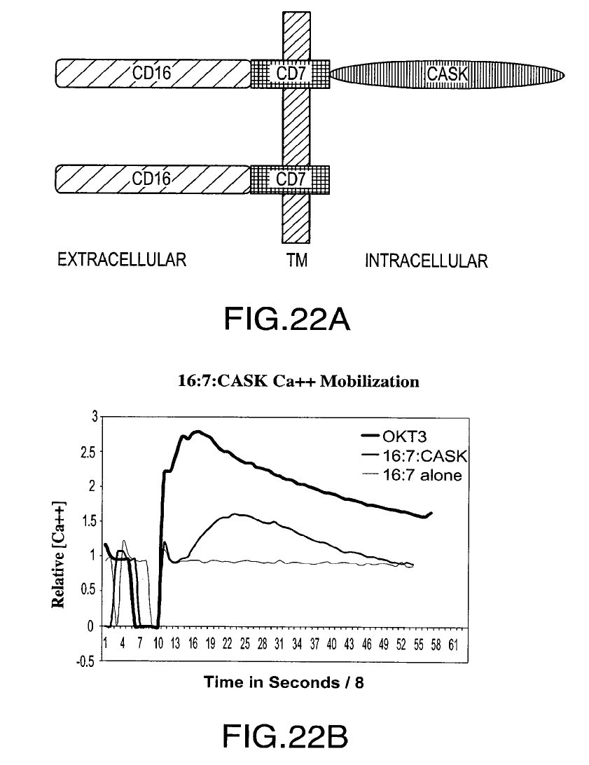

A schematic representation of the CD16: 7: CASK chimeric protein consisting of the extracellular domain of CD16, the transmembrane domain of CD7 linked to CASK is shown in FIG. 22A. The CD16: 7 chimera that was constructed lacked the membrane-linked CASK portion. Jurkat cells expressing the indicated chimeric proteins were loaded with a calcium fluorescent dye whose fluorescence properties are altered upon binding of free intracellular calcium. Cells were stimulated with OKT3 mAb (top tracing), or anti-CD16 antibody. As shown in FIG. 22B, while engagement of the CD16: 7: CASK chimera resulted in detectable mobilization of intracellular calcium (intermediate tracing), stimulation of the chimera lacking CASK sequences failed to do so (flat tracing). Thus, these results indicate that CASK is partially responsible or involved in T cell activation as measured by Ca+ flux. This could in part be due to the association with activated Ras, which is in the activation pathway.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

TABLE IV

| Protein | | |
|---|---|---|
| Name | Acc# or gi# | Reference |
| Akt | 18583311 | Direct Genbank submission |
| ankyrin | 178646 | Lambert et al. Proc. Natl. Acad. |
| | | Sci. 87: 1730-4 (1990) |
| Beta3- | 386833 | Kuppevelt et al. Proc. Natl. Acad. |
| integrin | | Sci. 86: 5415-18 (1989) |
| BLR-1 | 4502415 | Dobner et al. Eur. J. Immunol. 22: |
| | | 2795-99 (1992) |
| CaMKII | 7706286 | Lin et al. Proc. Natl. Acad. Sci. |
| | | 84: 5962-66 (1987) |
| Cask | 2641549 | Cohen et al. J. Cell Biol. 142: |
| | | 129-138 (1998)- |
| Cbl | 115855 | Blake et al. Oncogene 6: 653-7 |
| | | (1991) |
| CD16 | X16863 | Simmons, D., and Seed, B. Nature |
| | | 333: 568-70 (1988) |
| CD28 | J02988 | Aruffo, A. and Seed, B. Proc. Natl. |
| | | Acad. Sci. 84: 8573-77 (1987) |
| CD34 | M81104 | Simmons et al. J. Immunol. 148: |
| | | 267-71 (1992) |
| CD3zeta | J04132 | Weissman et al. Proc. Natl. Acad. |
| | | Sci. 85: 9709-13 (1988) |
| CD45 | Y00638 | Streuli et al. J. Exp. Med. 166: |
| | | 1548-66 (1987) |
| CD48 | X06341 | Killeen et al. EMBO J. 7: |
| | | 3087-91 (1988) |
| CD7 | X06180 | Aruffo, A. and Seed, B. EMBO J. 6: |
| | | 3313-16 (1987) |
| Cdc42 | 7662108 | Ishikawa et al. DNA Res. 4 (5), |
| | | 307-313 (1997) |
| Chapsyn | 1463026 | Kim et al. Neuron 17: 103-13 (1996) |
| CNK | 3930781 | Therrien et al. Cell 95: 343-53 |
| | | (1998) |
| CSK | 729887 | Brauninger et al. Oncogene 8: |
| | | 1365-9 (1993) |
| DNAM-1 | 1401185 | Shibuya et al. Immunity 4: 573-81 |
| | | (1996) |
| Dock2 | 18560620 | Direct Genbank submission |
| Dvl1 | 2291005 | Semenov, M. and Snyder, M. |
| | | Genomics 42: 302-10 (1997) |
| Dvl2 | 2291007 | Semenov, M. and Snyder, M. |
| | | Genomics 42: 302-10 (1997) |
| EBP-50 | 3220019 | Reczek et al. J. Cell Biol. 139: |
| | | 169-79 (1997) |
| FcERI | 232084 | Kuster et al. J. Biol. Chem. 267: |
| | | 12782-7 (1992) |
| Fyn | 4503823 | Kawakami et al. Mol. Cell. Biol. |
| | | 6: 4195-201 (1986) |
| GADS | 6685489 | Qiu et al. Biochem. Biophys. Res. |
| | | Comm. 253: 443-7 (1998) |
| GKAP | 18201963 | Satoh et al. Genes Cells 2: 415-24 |
| | | (1997) |
| Grip | 4539084 | Bruckner et al. Neuron 22: 511-24 |
| | | (1999) |
| hDLG/SAP97 | 4758162 | Lue et al. Proc. Natl. Acad. Sci. |
| | | 91: 9818-22 (1994) |
| IQGAP | 1170586 | Weissbach et al. J. Biol. Chem. |
| | | 269: 20517-21 (1994) |
| ITK | 585361 | Tanaka et al. FEBS Lett. 324: 1-5 |
| | | (1993) |
| KIAA0807 | 18547533 | Direct Genbank submission |
| KIAA1481 | 17443334 | Direct Genbank submission |
| LAT | 14194891 | Zhang et al. Cell 92: 83-92 (1998) |
| Lck | 66786 | Perlmutter et al. J. Cell. Biochem. |
| | | 38: 117-26 (1988) |
| LFA-1 | 1170591 | Larson et al. J. Cell Biol. 108: |
| | | 703-12 (1989) |
| LPAP | 1082575 | Schraven et al. J. Biol. Chem. 269: |
| | | 29102-111 (1994) |
| Neuroligin | 18595051 | Direct Genbank submission |
| PAG | 16753229 | Brdicka et al. J. Exp. Med. 191: |
| | | 1591-604 (2000) |
| PDZrhoGEF | 7662088 | Kourlas et al. Proc. Natl. Acad. |
| | | Sci. 97: 2145-2150 (2000) |
| Pick1 | 6691439 | Takeya et al. Biochem. Biophys. |
| | | Res. Comm. 267: 149-55 (2000) |
| PKCtheta | 423039 | Baier, G. J. Biol. Chem. 268: |
| | | 4997-5004 (1993) |
| PSD95 | 3318653 | Stathakis, D. Genomics 44: 71-82 |
| | | (1997) |
| PTEN | 5051943 | Direct Genbank submission |
| SHANK1 | 6049186 | Lim et al. J. Biol. Chem. 274: |
| | | 29510-8 (1999) |
| SHANK3 | 14779793 | Direct Genbank submission |
| Shroom | 7959222 | Direct Genbank submission |
| SLP76 | 5031855 | Jackman et al. J. Biol. Chem. 270: |
| | | 7029-32 (1995) |
| spectrin | 4507191 | Leto et al. Mol. Cell. Biol. 8 |
| | | (1), 1-9 (1988) |
| Syk | 1174527 | Law et al. J. Biol. Chem. 269: |
| | | 12310-9 (1994) |
| Tek | 14738136 | Ziegler et al. Oncogene 8 (3), |
| | | 663-670 (1993) |
| Tip1 | 14579004 | Reynaud et al. J. Biol. Chem. 275: |
| | | 33962-8 (2000) |
| Vav | 7108367 | Katzav et al. EMBO J. 8: 2283-90 |
| | | (1989) |
| VLA-2 | 4504743 | Takada and Hemler. J. Cell Biol. |
| | | 109 (1), 397-407 (1989) |
| WASP | 1722836 | Derry et al. Cell 78: 635-44 (1994) |
| ZAP-70 | 340038 | Chan et al. Cell 71: 649-662 (1992) |
| ZO-1 | 585098 | Willott et al. Proc. Natl. Acad. |
| | | Sci. 90: 7834-8 (1993) |

### REFERENCES

1. Klausner, R. D., A. M. Kleinfeld, R. L. Hoover, and M. J. Karnovsky. 1980. Lipid domains in membranes. Evidence derived from structural perturbations induced by free fatty acids and lifetime heterogeneity analysis. J Biol Chem 255, no. 4:1286.
2. Brown, D. A., and E. London. 2000. Structure and function of sphingolipid- and cholesterol-rich membrane rafts. J Biol Chem 275, no. 23:17221.
3. Brown, D. A., and E. London. 1998. Structure and origin of ordered lipid domains in biological membranes. J Membr Biol 164, no. 2:103.
4. Melkonian, K. A., A. G. Ostermeyer, J. Z. Chen, M. G. Roth, and D. A. Brown. 1999. Role of lipid modifications in targeting proteins to detergent-resistant membrane rafts. Many raft proteins are acylated, while few are prenylated. J Biol Chem 274, no. 6:3910.
5. Bunnell, S. C., M. Diehn, M. B. Yaffe, P. R. Findell, L. C. Cantley, and L. J. Berg. 2000. Biochemical interactions integrating Itk with the T cell receptor-initiated signaling cascade. J Biol Chem 275, no. 3:2219.
6. Sheets, E. D., D. Holowka, and B. Baird. 1999. Critical role for cholesterol in Lyn-mediated tyrosine phosphorylation of FcepsilonRI and their association with detergent-resistant membranes. J Cell Biol 145, no. 4:877.
7. Zhang, W., J. Sloan-Lancaster, J. Kitchen, R. P. Trible, and L. E. Samelson. 1998. LAT: the ZAP-70 tyrosine kinase substrate that links T cell receptor to cellular activation. Cell 92, no. 1:83.
8. Zhang, W., R. P. Trible, and L. E. Samelson. 1998. LAT palmitoylation: its essential role in membrane microdomain targeting and tyrosine phosphorylation during T cell activation. Immunity 9, no. 2:239.
9. Kabouridis, P. S., A. I. Magee, and S. C. Ley. 1997. S-acylation of LCK protein tyrosine kinase is essential for its signalling function in T lymphocytes. Embo J 16, no. 16:4983.
10. Cheng, P. C., M. L. Dykstra, R. N. Mitchell, and S. K. Pierce. 1999. A role for lipid rafts in B cell antigen receptor signaling and antigen targeting. J Exp Med 190, no. 11:1549.
11. Field, K. A., D. Holowka, and B. Baird. 1999. Structural aspects of the association of FcepsilonRI with detergent-resistant membranes. J Biol Chem 274, no. 3:1753.
12. Montixi, C., C. Langlet, A. M. Bernard, J. Thimonier, C. Dubois, M. A. Wurbel, J. P. Chauvin, M. Pierres, and H. T. He. 1998. Engagement of T cell receptor triggers its recruitment to low-density detergent-insoluble membrane domains. Embo J 17, no. 18:5334.
13. Xavier, R., T. Brennan, Q. Li, C. McCormack, and B. Seed. 1998. Membrane compartmentation is required for efficient T cell activation. Immunity 8, no. 6:723.
14. Janes, P. W., S. C. Ley, and A. I. Magee. 1999. Aggregation of lipid rafts accompanies signaling via the T cell antigen receptor. J Cell Biol 147, no. 2:447.
15. Lou, Z., D. Jevremovic, D. D. Billadeau, and P. J. Leibson. 2000. A balance between positive and negative signals in cytotoxic lymphocytes regulates the polarization of lipid rafts during the development of cell-mediated killing. J Exp Med 191, no. 2:347.
16. Manes, S., E. Mira, C. Gomez-Mouton, R. A. Lacalle, P. Keller, J. P. Labrador, and A. C. Martinez. 1999. Membrane raft microdomains mediate front-rear polarity in migrating cells. Embo J 18, no. 22:6211.
17. Songyang, Z., A. S. Fanning, C. Fu, J. Xu, S. M. Marfatia, A. H. Chishti, A. Crompton, A. C. Chan, J. M. Anderson, and L. C. Cantley. 1997. Recognition of unique carboxyl-terminal motifs by distinct PDZ domains. Science 275, no. 5296:73.
18. Saras, J., and C. H. Heldin. 1996. PDZ domains bind carboxy-terminal sequences of target proteins. Trends Biochem Sci 21, no. 12:455.
19. Kawabuchi, M., Y. Satomi, T. Takao, Y. Shimonishi, S, Nada, K. Nagai, A. Tarakhovsky, and M. Okada. 2000. Transmembrane phosphoprotein Cbp regulates the activities of Src-family tyrosine kinases. Nature 404, no. 6781:999.
20. Brdicka, T., D. Pavlistova, A. Leo, E. Bruyns, V. Korinek, P. Angelisova, J. Scherer, A. Shevchenko, I. Hilgert, J. Cerny, K. Drbal, Y. Kuramitsu, B. Komacker, V. Horejsi, and B. Schraven. 2000. Phosphoprotein associated with glycosphingolipid-enriched microdomains (PAG), a novel ubiquitously expressed transmembrane adaptor protein, binds the protein tyrosine kinase csk and is involved in regulation of T cell activation. J Exp Med 191, no. 9:1591.
21. Sicheri, F., 1. Moarefi, and J. Kuriyan. 1997. Crystal structure of the Src family tyrosine kinase Hck. Nature 385, no. 6617:602.
22. Plas, D. R., and M. L. Thomas. 1998. Negative regulation of antigen receptor signaling in lymphocytes. J Mol Med 76, no. 8:589.
23. Schraven, B., D. Schoenhaut, E. Bruyns, G. Koretzky, C. Eckerskorn, R. Wallich, H. Kirchgessner, P. Sakorafas, B. Labkovsky, S. Ratnofsky, and et al. 1994. LPAP, a novel 32-kDa phosphoprotein that interacts with CD45 in human lymphocytes. J Biol Chem 269, no. 46:29102.
24. Matsuda, A., S. Motoya, S. Kimura, R. McInnis, A. L. Maizel, and A. Takeda. 1998. Disruption of lymphocyte function and signaling in CD45-associated protein-null mice. J Exp Med 187, no. 11:1863.
25. Johnson, K. G., S. K. Bromley, M. L. Dustin, and M. L. Thomas. 2000. A supramolecular basis for CD45 tyrosine phosphatase regulation in sustained T cell activation. Proc Natl Acad Sci USA 97, no. 18:10138.
26. Flanagan, W. M., B. Corthesy, R. J. Bram, and G. R. Crabtree. 1991. Nuclear association of a T-cell transcription factor blocked by FK-506 and cyclosporin A. Nature 352, no. 6338:803.
27. Woodrow, M., N. A. Clipstone, and D. Cantrell. 1993. p21ras and calcineurin synergize to regulate the nuclear factor of activated T cells. J Exp Med 178, no. 5:1517.
28. Pan, G., J. H. Bauer, V. Haridas, S. Wang, D. Liu, G. Yu, C. Vincenz, B. B. Aggarwal, J. Ni, and V. M. Dixit. 1998. Identification and functional characterization of DR6, a novel death domain-containing TNF receptor. FEBS Lett 431, no. 3:351.
29. Yang, W. C., Y. Collette, J. A. Nunes, and D. Olive. 2000. Tec kinases: a family with multiple roles in immunity. Immunity 12, no. 4:373.
30. Dustin, M. L., and A. C. Chan. 2000. Signaling takes shape in the immune system. Cell 103, no. 2:283.
31. Perez-Villar, J. J., and S. B. Kanner. 1999. Regulated association between the tyrosine kinase Emt/Itk/Tsk and phospholipase-C gamma 1 in human T lymphocytes. J Immunol 163, no. 12:6435.
32. Liu, K. Q., S. C. Bunnell, C. B. Gurniak, and L. J. Berg. 1998. T cell receptor-initiated calcium release is uncoupled from capacitative calcium entry in Itk-deficient T cells. J Exp Med 187, no. 10:1721.
33. Liao, X. C., and D. R. Littman. 1995. Altered T cell receptor signaling and disrupted T cell development in mice lacking Itk. Immunity 3, no. 6:757.
34. Fowell, D. J., K. Shinkai, X. C. Liao, A. M. Beebe, R. L. Coffman, D. R. Littman, and R. M. Locksley. 1999. Impaired NFATc translocation and failure of Th2 development in Itk-deficient CD4+ T cells. Immunity 11, no. 4:399.
35. Chung, H. J., X. J., S. R. H., Z. X., and H. R. L. 2000. Phosphorylation of the AMPA receptor subunit GluR2 differentially regulates its interaction with PDZ domain-containing proteins. Journal of Neuroscience 20, no. 19:7258.
36. Hanada, T., L. Lin, K. G. Chandy, S. S. Oh, and A. H. Chishti. 1997. Human homologue of the Drosophila discs large tumor suppressor binds to p56lck tyrosine kinase and Shaker type Kv1.3 potassium channel in T lymphocytes. J Biol Chem 272, no. 43:26899.
37. Hanada, T., L. Lin, E. V. Tibaldi, E. L. Reinherz, and A. H. Chishti. 2000. GAKIN, a novel kinesin-like protein associates with the human homologue of the Drosophila discs large tumor suppressor in T lymphocytes. J Biol Chem 275, no. 37:28774.
38. Reynaud, C., S. Fabre, and P. Jalinot. 2000. The PDZ protein TIP-1 interacts with the rho effector rhotekin and is involved in rho signaling to the serum response element. J Biol Chem 275, no. 43:33962.
39. Mosmann, T. R., H. Cherwinski, M. W. Bond, M. A. Giedlin, and R. L. Coffman. 1986. Two types of murine helper T cell clone. I. Definition according to profiles of lymphokine activities and secreted proteins. J Immunol 136, no. 7:2348.
40. Seder, R. A., and W. E. Paul. 1994. Acquisition of lymphokine-producing phenotype by CD4+ T cells. Annu Rev Immunol 12, no. 18:635.
41. Locksley, R. M., and P. Scott. 1991. Helper T-cell subsets in mouse leishmaniasis: induction, expansion and effector function. Immunol Today 12, no. 3:A58.
42. Acuto, O., and Cantrell, D. (2000). T cell activation and the cytoskeleton, Annu Rev Immunol 18, 165-84.
43. Bi, K., Tanaka, Y., Coudronniere, N., Sugie, K., Hong, S., van Stipdonk, M. J., and Altman, A. (2001). Antigen-induced translocation of PKC-straight theta to membrane rafts is required for T cell activation, Nat Immunol 2, 556-63.
44. Bilder, D., Li, M., and Perrimon, N. (2000). Cooperative regulation of cell polarity and growth by Drosophila tumor suppressors, Science 289, 113-6.
45. Bromley, S. K., Burack, W. R., Johnson, K. G., Somersalo, K., Sims, T. N., Sumen, C., Davis, M. M., Shaw, A. S., Allen, P. M., and Dustin, M. L. (2001). The immunological synapse, Annu Rev Immunol 19, 375-96.
46. Bunnell, S. C., Kapoor, V., Trible, R. P., Zhang, W., and Samelson, L. E. (2001). Dynamic actin polymerization drives T cell receptor-induced spreading: a role for the signal transduction adaptor LAT, Immunity 14, 315-29.
47. Butz, S., Okamoto, M., and Sudhof, T. C. (1998). A tripartite protein complex with the potential to couple synaptic vesicle exocytosis to cell adhesion in brain, Cell 94, 773-82.
48. Chen, F., Ma, L., Parrini, M. C., Mao, X., Lopez, M., Wu, C., Marks, P. W., Davidson, L., Kwiatkowski, D. J., Kirchhausen, T., et al. (2000). Cdc42 is required for PIP(2)-induced actin polymerization and early development but not for cell viability, Curr Biol 10, 758-65.
49. Christopherson, K. S., Hillier, B. J., Lim, W. A., and Bredt, D. S. (1999). PSD-95 assembles a ternary complex with the N-methyl-D-aspartic acid receptor and a bivalent neuronal NO synthase PDZ domain, J Biol Chem 274, 27467-73.
50. Coudronniere, N., Villalba, M., Englund, N., and Altman, A. (2000). NF-kappa B activation induced by T cell receptor/CD28 costimulation is mediated by protein kinase C-theta, Proc Natl Acad Sci USA 97, 3394-9.
51. Dong, H., O'Brien, R. J., Fung, E. T., Lanahan, A. A., Worley, P. F., and Huganir, R. L. (1997). GRIP: a synaptic PDZ domain-containing protein that interacts with AMPA receptors, Nature 386, 279-84.
52. Dustin, M. L., Olszowy, M. W., Holdorf, A. D., Li, J., Bromley, S., Desai, N., Widder, P., Rosenberger, F., van der Merwe, P. A., Allen, P. M., and Shaw, A. S. (1998). A novel adaptor protein orchestrates receptor patterning and cytoskeletal polarity in T-cell contacts, Cell 94, 667-77.
53. Fraser, J. D., Newton, M. E., and Weiss, A. (1992). CD28 and T cell antigen receptor signal transduction coordinately regulate interleukin 2 gene expression in response to superantigen stimulation, J Exp Med 175, 1131-4.
54. Goldsmith, M. A., and Weiss, A. (1987). Isolation and characterization of a T-lymphocyte somatic mutant with altered signal transduction by the antigen receptor, Proc Natl Acad Sci USA 84, 6879-83.
55. Grakoui, A., Bromley, S. K., Sumen, C., Davis, M. M., Shaw, A. S., Allen, P. M., and Dustin, M. L. (1999). The immunological synapse: a molecular machine controlling T cell activation, Science 285, 221-7.
56. Hanada, T., Lin, L., Chandy, K. G., Oh, S. S., and Chishti, A. H. (1997). Human homologue of the Drosophila discs large tumor suppressor binds to p56lck tyrosine kinase and Shaker type Kv1.3 potassium channel in T lymphocytes, J Biol Chem 272, 26899-904.
57. Hanada, T., Lin, L., Tibaldi, E. V., Reinherz, E. L., and Chishti, A. H. (2000). GAKIN, a novel kinesin-like protein associates with the human homologue of the Drosophila discs large tumor suppressor in T lymphocytes, J Biol Chem 275, 28774-84.
58. Hata, Y., Butz, S., and Sudhof, T. C. (1996). CASK: a novel dlg/PSD95 homolog with an N-terminal calmodulin-dependent protein kinase domain identified by interaction with neurexins, J Neurosci 16, 2488-94.
59. Kawabuchi M, et al (2000): Transmembrane phosphoprotein Cbp regulates the activities of Src-family tyrosine kinases. Nature, April 27; 404(6781):999-1003
60. Khan, A. A., Bose, C., Yam, L. S., Soloski, M. J., and Rupp, F. (2001). Physiological regulation of the immunological synapse by agrin, Science 292, 1681-6.
61. Lim, S., Naisbitt, S., Yoon, J., Hwang, J. I., Suh, P. G., Sheng, M., and Kim, E. (1999). Characterization of the Shank family of synaptic proteins. Multiple genes, alternative splicing, and differential expression in brain and development, J Biol Chem 274, 29510-8.
62. Lue, R. A., Marfatia, S. M., Branton, D., and Chishti, A. H. (1994). Cloning and characterization of hdlg: the human homologue of the Drosophila discs large tumor suppressor binds to protein 4.1, Proc Natl Acad Sci USA 91, 9818-22.
63. Monks, C. R., Freiberg, B. A., Kupfer, H., Sciaky, N., and Kupfer, A. (1998). Three-dimensional segregation of supramolecular activation clusters in T cells, Nature 395, 82-6.
64. Monks, C. R., Kupfer, H., Tamir, 1., Barlow, A., and Kupfer, A. (1997). Selective modulation of protein kinase C-theta during T-cell activation, Nature 385, 83-6.
65. Moores, S. L., Selfors, L. M., Fredericks, J., Breit, T., Fujikawa, K., Alt, F. W., Brugge, J. S., and Swat, W. (2000). Vav family proteins couple to diverse cell surface receptors, Mol Cell Biol 20, 6364-73.
66. Muller, B. M., Kistner, U., Veh, R. W., Cases-Langhoff, C., Becker, B., Gundelfinger, E. D., and Garner, C. C. (1995). Molecular characterization and spatial distribution of SAP97, a novel presynaptic protein homologous to SAP90 and the Drosophila discs-large tumor suppressor protein, J Neurosci 15, 2354-66.
67. Naisbitt, S., et al. (1999) Shank, a novel family of postsynaptic density proteins that binds to the NMDA receptor/PSD-95/GKAP complex and cortactin. Neuron 23(3):569-82.
68. Rohatgi, R., Ma, L., Miki, H., Lopez, M., Kirchhausen, T., Takenawa, T., and Kirschner, M. W. (1999). The interaction between N-WASP and the Arp2/3 complex links Cdc42-dependent signals to actin assembly, Cell 97, 221-31.
69. Romeo, C., Seed, B. (1991) Cellular immunity to HIV activated by CD4 fused to T cell or Fc receptor polypeptides, Cell 64,1037-46.
70. Shapiro, V. S., Mollenauer, M. N., and Weiss, A. (1998). Nuclear factor of activated T cells and AP-1 are insufficient for IL-2 promoter activation: requirement for CD28 up-regulation of RE/AP, J Immunol 161, 6455-8.
71. Sheng, M., and Sala, C. (2001). Pdz domains and the organization of supramolecular complexes, Annu Rev Neurosci 24, 1-29.
72. Songyang, Z., Fanning, A. S., Fu, C., Xu, J., Marfatia, S. M., Chishti, A. H., Crompton, A., Chan, A. C., Anderson, J. M., and Cantley, L. C. (1997). Recognition of unique carboxyl-terminal motifs by distinct PDZ domains, Science 275, 73-7.
73. Staudinger, J., Zhou, J., Burgess, R., Elledge, S. J., and Olson, E. N. (1995). PICK1: a perinuclear binding protein and substrate for protein kinase C isolated by the yeast two-hybrid system, J Cell Biol 128, 263-71.
74. Straus, D. B., and Weiss, A. (1992). Genetic evidence for the involvement of the lck tyrosine kinase in signal transduction through the T cell antigen receptor, Cell 70, 585-93.
75. Sun, Z., Arendt, C. W., Ellmeier, W., Schaeffer, E. M., Sunshine, M. J., Gandhi, L., Annes, J., Petrzilka, D., Kupfer, A., Schwartzberg, P. L., and Littman, D. R. (2000). PKC-theta is required for TCR-induced NF-kappaB activation in mature but not immature T lymphocytes, Nature 404, 402-7.
76. Therrien, M., Wong, A. M., and Rubin, G. M. (1998). CNK, a RAF-binding multidomain protein required for RAS signaling, Cell 95, 343-53.
77. Thomas, U., Ebitsch, S., Gorczyca, M., Koh, Y. H., Hough, C. D., Woods, D., Gundelfinger, E. D., and Budnik, V. (2000). Synaptic targeting and localization of discs-large is a stepwise process controlled by different domains of the protein, Curr Biol 10, 1108-17.
78. Ting, A. T., Pimentel-Muinos, F. X., and Seed, B. (1996). RIP mediates tumor necrosis factor receptor 1 activation of NF-kappaB but not Fas/APO-1-initiated apoptosis, Embo J 15, 6189-96.
79. Tsunoda, S., Sierralta, J., Sun, Y., Bodner, R., Suzuki, E., Becker, A., Socolich, M., and Zuker, C. S. (1997). A multivalent PDZ-domain protein assembles signalling complexes in a G-protein-coupled cascade, Nature 388, 243-9.
80. Williams, B. L., Schreiber, K. L., Zhang, W., Wange, R. L., Samelson, L. E., Leibson, P. J., and Abraham, R. T. (1998). Genetic evidence for differential coupling of Syk family kinases to the T-cell receptor: reconstitution studies in a ZAP-70-deficient Jurkat T-cell line, Mol Cell Biol 18, 1388-99.
81. Wu, H., Reuver, S. M., Kuhlendahl, S., Chung, W. J., and Garner, C. C. (1998). Subcellular targeting and cytoskeletal attachment of SAP97 to the epithelial lateral membrane, J Cell Sci 111, 2365-76.
82. Wu, J., Katzav, S., and Weiss, A. (1995). A functional T-cell receptor signaling pathway is required for p95vav activity, Mol Cell Biol 15, 4337-46.
83. Wulfing, C., and Davis, M. M. (1998). A receptor/cytoskeletal movement triggered by costimulation during T cell activation, Science 282, 2266-9.
84. Xavier, R., Brennan, T., Li, Q., McCormack, C., and Seed, B. (1998). Membrane compartmentation is required for efficient T cell activation, Immunity 8, 723-32.

1. A method for screening a compound to determine whether the compound modulates immune cell signaling, the method comprising identifying a compound that modulates interaction between a PDZ protein and a PDZ ligand protein (a PL protein), wherein the PDZ protein and the PL protein are proteins which in an immune cell can interact with one another to affect the composition and/or distribution of lipid rafts in the immune cell.

2. The method of claim 1, wherein identifying comprises
(a) contacting a PDZ domain polypeptide that comprises at least a partial sequence of the PDZ protein and a PL domain polypeptide that comprises at least a partial sequence of the PL protein in the presence of the compound; and
(b) determining whether there is a statistically significant difference in the amount of complex formed between the PDZ domain polypeptide and the PL domain polypeptide in the presence of the compound as compared to the amount of the complex formed in the absence of the compound, a statistically significant difference being an indication that the compound is a modulator of immune cell signaling.

3. The method of claim 1, wherein the PDZ protein is selected from the group consisting of hDlg, SHANK1, SHANK3, EBP-50, CASK, KIAA0807, TIP1, PSD-95, Pick1, CNK, GRIP and DVL-2.

4. The method of claim 1, wherein the PL protein is selected from the group consisting of PAG, LPAP, ITK, DNAM-1, Shroom, PTEN, BLR-1, fyn and Na+/Pi transporter.

5. The method of claim 1, wherein
(a) the PDZ protein is SHANK1 or SHANK3 and the PL protein is PAG, LPAP, ITK, DNAM-1, Shroom, PTEN, BLR-1 or fyn;
(b) the PDZ protein is TIP1 and the PL protein is LPAP or PAG;
(c) the PDZ protein is KIAA0807 and the PL protein is PAG or LPAP;
(d) the PDZ protein is EBP-50 and the PL is PAG or LPAP or BLR-1; or
(e) the PDZ protein is SHANK3 or EBP-50 and the PL protein is Na+/Pi transporter.

6. A method for modulating immune cell signaling, the method comprising modulating an interaction between a PDZ protein and a PDZ ligand protein (a PL protein), which interaction affects the composition and/or distribution of lipid rafts in an immune cell, and whereby such modulation alters immune cell signaling.

7. The method of claim 6, wherein the PDZ protein is selected from the group consisting of hDlg, SHANK1, SHANK3, EBP-50, CASK, KIAA0807, TIP1, PSD-95, Pick1, CNK, GRIP and DVL-2.

8. The method of claim 6, wherein the PL protein is selected from the group consisting of PAG, LPAP, ITK, DNAM-1, Shroom, PTEN, BLR-1, fyn and Na+/Pi transporter.

9. The method of claim 6, wherein
(a) the PDZ protein is SHANK1 or SHANK3 and the PL protein is PAG, LPAP, ITK, DNAM-1, Shroom, PTEN, BLR-1 or fyn;
(b) the PDZ protein is TIP1 and the PL protein is LPAP or PAG;
(c) the PDZ protein is KIAA0807 and the PL protein is PAG or LPAP;
(d) the PDZ protein is EBP-50 and the PL is PAG or LPAP or BLR-1; or
(e) the PDZ protein is SHANK3 or EBP-50 and the PL protein is Na+/Pi transporter.

10. The method of claim 6, wherein modulating comprises contacting an immune cell with a compound that inhibits or enhances interaction between the PDZ protein and the PL protein.

11. The method of claim 10, wherein the compound includes a tetrazole moiety.

12. The method of claim 10, wherein contacting comprises administering the compound to a patient having an immune disorder, the compound being administered in an amount effective to treat the immune disorder.

13. The method of claim 12, wherein the immune disorder is an autoimmune disorder.

14. The method of claim 12, wherein the immune disorder is selected from the group consisting of systemic lupus erythematosus (SLE), multiple sclerosis, diabetes mellitus, rheumatoid arthritis, inflammatory bowel syndrome, psoriasis, scleroderma, inflammatory myopathies, autoimmune hemolytic anemia, graves disease, Wiskott-Aldrich syndrome, lymphoma, leukemia, severe combined immunodeficiency syndrome (SCID) and acquired immunodeficiency syndrome (AIDS).

15. The method of claim 10, wherein the compound enhances the interaction between the PDZ protein and the PL protein.

16. The method of claim 10, wherein the compound inhibits the interaction between the PDZ protein and the PL protein.

17. The method of claim 16, wherein the compound is
(a) a polypeptide or fusion polypeptide comprising a sequence that is from 2 to about 20 residues of the C-terminal sequence of the PL protein;
(b) a polypeptide or fusion polypeptide comprising a sequence that is from 2 to about 100 residues of the PDZ domain of the PDZ protein; or
(c) a small molecule mimetic of the polypeptide or fusion polypeptide of section (a) or (b).

18. The method of claim 6, wherein the immune cell is a T-cell.

19. The method of claim 6, wherein the immune cell is a B-cell.

20. The method of claim 6, wherein the immune cell is a monocyte/macrophage.

21. A modulator of binding of a PDZ protein and a PDZ ligand protein (a PL protein), wherein the modulator inhibits or enhances binding of a PDZ domain polypeptide and a PL domain polypeptide, and wherein
(a) the PDZ domain polypeptide comprises at least a partial sequence of the PDZ protein and the PL domain polypeptide comprises at least a partial sequence of the PL protein; and
(b) the PDZ protein and the PL protein are proteins which in an immune cell can interact with o

22. The modulator of claim 21, wherein the modulator is formulated as a pharmaceutical composition that comprises the modulator and a pharmaceutically acceptable carrier.

23. The modulator of claim 21, wherein the modulator inhibits binding of the PDZ domain polypeptide and the PL domain polypeptide.

24. The modulator of claim 21, wherein the modulator enhances binding of the PDZ domain polypeptide and the PL domain polypeptide.

25. The modulator of claim 21, wherein the modulator is
(a) a polypeptide or fusion polypeptide comprising a sequence that is from 2 to about 20 residues of a C-terminal sequence of the PL protein;
(b) a polypeptide or fusion polypeptide comprising a sequence that is from 2 to about 100 residues of the PDZ domain of the PDZ protein; or
(c) a peptide or small molecule mimetic of the polypeptide or fusion polypeptide of section (a) or (b).

26. The modulator of claim 21, wherein the PDZ protein is selected from the group consisting of hDlg, SHANK1, SHANK3, EBP-50, CASK, KIAA0807, TIP1, PSD-95, Pick1, CNK, GRIP and DVL-2.

27. The method of claim 21, wherein the PL protein is selected from the group consisting of PAG, LPAP, ITK, DNAM-1, Shroom, PTEN, BLR-1, fyn and Na+/Pi transporter.

28. The method of claim 21, wherein
(a) the PDZ protein is SHANK1 or SHANK3 and the PL protein is PAG, LPAP, ITK, DNAM-1, Shroom, PTEN, BLR-1 or fyn;
(b) the PDZ protein is TIP1 and the PL protein is LPAP or PAG;
(c) the PDZ protein is KIAA0807 and the PL protein is PAG or LPAP;
(d) the PDZ protein is EBP-50 and the PL is PAG or LPAP or BLR-1; or
(e) the PDZ protein is SHANK3 or EBP-50 and the PL protein is Na+/Pi transporter.

29. The use of a modulator of the binding of a PDZ protein and a PDZ ligand protein (a PL protein) to treat an immune disorder, wherein the PDZ protein and the PL protein are proteins which in an immune cell can interact with one another to affect the composition and/or distribution of lipid rafts in the immune cell.

30. The method of claim 29, wherein the PDZ protein is selected from the group consisting of hDlg, SHANK1, SHANK3, EBP-50, CASK, KIAA0807, TIP1, PSD-95, Pick1, CNK, GRIP and DVL-2.

31. The method of claim 29, wherein the PL protein is selected from the group consisting of PAG, LPAP, ITK, DNAM-1, Shroom, PTEN, BLR-1, fyn and Na+/Pi transporter.

32. The method of claim 29, wherein
(a) the PDZ protein is SHANK1 or SHANK3 and the PL protein is PAG, LPAP, ITK, DNAM-1, Shroom, PTEN, BLR-1 or fyn;
(b) the PDZ protein is TIP1 and the PL protein is LPAP or PAG;
(c) the PDZ protein is KIAA0807 and the PL protein is PAG or LPAP;
(d) the PDZ protein is EBP-50 and the PL is PAG or LPAP or BLR-1; or
(e) the PDZ protein is SHANK3 or EBP-50 and the PL protein is Na+/Pi transporter.

33. The use of a modulator of the binding of a PDZ protein and a PDZ ligand protein (a PL protein) in the preparation of a medicament for treatment of an immune disease, wherein the PDZ protein and the PL protein are proteins which in an immune cell can interact with one another to affect the composition and/or distribution of lipid rafts in the immune cell.

34. The method of claim 33, wherein the PDZ protein is selected from the group consisting of hDlg, SHANK1, SHANK3, EBP-50, CASK, KIAA0807, TIP1, PSD-95, Pick1, CNK, GRIP and DVL-2.

35. The method of claim 33, wherein the PL protein is selected from the group consisting of PAG, LPAP, ITK, DNAM-1, Shroom, PTEN, BLR-1, fyn and Na+/Pi transporter.

36. The method of claim 33, wherein
(a) the PDZ protein is SHANK1 or SHANK3 and the PL protein is PAG, LPAP, ITK, DNAM-1, Shroom, PTEN, BLR-1 or fyn;
(b) the PDZ protein is TIP1 and the PL protein is LPAP or PAG;
(c) the PDZ protein is KIAA0807 and the PL protein is PAG or LPAP;
(d) the PDZ protein is EBP-50 and the PL is PAG or LPAP or BLR-1; or
(e) the PDZ protein is SHANK3 or EBP-50 and the PL protein is Na+/Pi transporter.
